SEQUENCE LISTING (104 sequences)

SEQ ID NO: 1 - protein, 53 aa, Arabidopsis thaliana
GDDKDSDDLNLFLNAIGEAGDEEGPTSFNDIDFLTFDDEDLHNPFQDCETSPI

SEQ ID NO: 2 - protein, 53 aa, Arabidopsis thaliana
MMSDVDIFGNGGITFDDFLYIMAQNTSQESASDELIEVFRVFDRDGDGLISQL

SEQ ID NO: 3 - protein, 53 aa, Arabidopsis thaliana
TSNQSGATSPTVFSGEFADDVDWSDENWPELEFRSAEDEAWYAVEFSDICDAL

SEQ ID NO: 4 - protein, 53 aa, Arabidopsis thaliana
ASDMSGMVMDTSVLDSAFTSDVGPDGEGAGNSRDSLRSFDQIPWNFSLSDLTA

SEQ ID NO: 5 - protein, 53 aa, Arabidopsis thaliana
PKNLEFYIDEEDCHLIPVEFYKPSEEVREISDINGDFILDFGVEHDFTAAAET

SEQ ID NO: 6 - protein, 53 aa, Arabidopsis thaliana
ALSKWASEGYMPTFDEYMEVGEVTGGMDDFALYSFIAMEDCDEKPLYEWFDSK

SEQ ID NO: 7 - protein, 53 aa, Saccharomyces cerevisiae
DGERNSNVRESAQGKALMTSEQNSNRYWNSFHDEDDWNLFNGMELESNGVVTF

SEQ ID NO: 8 - protein, 53 aa, Arabidopsis thaliana
CPSQDVVIHDCGEIPEGADDGICDFFKDGDVYPDWPIDLNESPAELSWWMETV

SEQ ID NO: 9 - protein, 53 aa, Saccharomyces cerevisiae
EPSMQDFDPNFEGDLYYLPKMDSSMNSANSDSNATEKRFIYGGYDDFLQPSIE

SEQ ID NO: 10 - protein, 53 aa, Saccharomyces cerevisiae
CANIPEFDSFYENENINYNLESFAPLNCDVNSPFLPINNNDINVNAYGDENLT

SEQ ID NO: 11 - protein, 53 aa, Saccharomyces cerevisiae
VDIFGNGGITFDDFLYIMAQNTSQESASDELIEVFRVFDRDGDGLISQLELGE

SEQ ID NO: 12 - protein, 53 aa, Arabidopsis thaliana
NEISSKANDDVLLDFDERDDVTNTNAGMLNTLTTLGDLDDLFDFGPSEDATQI

SEQ ID NO: 13 - protein, 53 aa, Saccharomyces cerevisiae
VNEDLQFDGFGADVESEFSVLNHMMEFNGYRSDRLEFDELEDDVSVIPLKGVN

SEQ ID NO: 14 - protein, 53 aa, Arabidopsis thaliana
RFYAELHIDDPIVTEYFKNQNTASIAELNSLQDIYDYLEFKYANEINEMFINH

SEQ ID NO: 15 - protein, 53 aa, Arabidopsis thaliana
IPDAENDLSFFDNGDKEKNDLFYGWGDIGNFEDVDNMLRSCDSTFGLDSLNNE

SEQ ID NO: 16 - protein, 53 aa, Saccharomyces cerevisiae
TSDVDGDGFIDFEEFLKLMEGEDGSDEERRKELKEAFGMYVMEGEEFITAASL

SEQ ID NO: 17 - protein, 53 aa, Arabidopsis thaliana
LSAEDEEEILAEFDNLESLLIVEDMPEVPTTELMPEEPEKMDLPDVPTKAPVA

SEQ ID NO: 18 - protein, 53 aa, Saccharomyces cerevisiae
HTQYDVEEEDMEVSAMLQDGKISMNEIFFEEENFQDINKILEFDNDFVAEFCS

SEQ ID NO: 19 - protein, 53 aa, Saccharomyces cerevisiae
DESEPLDLSHLQIPDGLGGPDDFDTQAGDLSSWLNIDDDALPDTDDLLGLQIP

SEQ ID NO: 20 - protein, 53 aa, Arabidopsis thaliana
IWGFEDHVSNYGGLDFGSGVGDGGDYVAVEGLFEFSDDCFDSGDLFSWRSESL

SEQ ID NO: 21 - protein, 53 aa, Saccharomyces cerevisiae
LKEDTGSTLYEFAMKLEDLNEPLSPWISSATGLEFFSEWENIPSELLKNLKPF

SEQ ID NO: 22 - protein, 53 aa, Saccharomyces cerevisiae
TSSAVTLPPTEEIDPMQGLSMDDEMKDVGFLPPIVSLDEFMESLNSEPPFGSP

SEQ ID NO: 23 - protein, 53 aa, Saccharomyces cerevisiae
LEDDTLDMEFDNHTRSEEDITLTDQIPTGIDPYVAVTFDEDIISESIPMDVDQ

SEQ ID NO: 24 - protein, 53 aa, Saccharomyces cerevisiae
QDGERNSNVRESAQGKALMTSEQNSNRYWNSFHDEDDWNLFNGMELESNGVVT

SEQ ID NO: 25 - protein, 53 aa, Arabidopsis thaliana
MSDVDIFGNGGITFDDFLYIMAQNTSQESASDELIEVFRVFDRDGDGLISQLE

SEQ ID NO: 26 - protein, 53 aa, Arabidopsis thaliana
ISWDEFAEAIRAFSPSITSEEIDNMFREIDVDGDNQIDVAEYASCLMLGGEGN

SEQ ID NO: 27 - protein, 53 aa, Arabidopsis thaliana
REVEEVVKTSDVDGDGFIDFEEFLKLMEGEDGSDEERRKELKEAFGMYVMEGE

SEQ ID NO: 28 - protein, 53 aa, Saccharomyces cerevisiae
LGIGWTSTMLSYERASWTDEFLNTSPSPEVFTLPEEQSGMAWEWHDKDWMLDL

SEQ ID NO: 29 - protein, 53 aa, Arabidopsis thaliana
DEIDMDDLVHFSPSIEFADTQLKSSGDFQLDDSWSSKDHEIFHFDPVTEFSDA

SEQ ID NO: 30 - protein, 53 aa, Saccharomyces cerevisiae
TFFLDDALSVLNSDKNSHLLSAVKRDFKDEDKVSLDEAIDLAWTNDEFDCLVD

SEQ ID NO: 31 - protein, 53 aa, Arabidopsis thaliana
MQDFDPNFEGDLYYLPKMDSSMNSANSDSNATEKRFIYGGYDDFLQPSIENSQ

SEQ ID NO: 32 - protein, 53 aa, Arabidopsis thaliana
SGEELQSCVSLLGGALSSREVEEVVKTSDVDGDGFIDFEEFLKLMEGEDGSDE

SEQ ID NO: 33 - protein, 53 aa, Saccharomyces cerevisiae
VEMKWDFDSLSSSDYIIENNINLDALAEDNNEWATAQHDLFNYAYPDEDSYYF

SEQ ID NO: 34 - protein, 53 aa, Arabidopsis thaliana
DNQEIDFLDLFSPTFKFNFASTTTDWQPIVAGPDECDESVSDLLAEVEAMESQ

SEQ ID NO: 35 - protein, 53 aa, Arabidopsis thaliana
EELQSCVSLLGGALSSREVEEVVKTSDVDGDGFIDFEEFLKLMEGEDGSDEER

SEQ ID NO: 36 - protein, 53 aa, Saccharomyces cerevisiae
ENVVFITSTAGQGEFPQDGKSFWEALKNDTDLDLASLNVAVFGLGDSEYWPRK

SEQ ID NO: 37 - protein, 53 aa, Saccharomyces cerevisiae
YFEEKRSLEDLWKVAFPVGTEWDQLDALYEFNWDFQNLEEALEEGGKLYGKKV

SEQ ID NO: 38 - protein, 53 aa, Saccharomyces cerevisiae
VFDFPGKDLQREEVIDLLDQQGFIPDDLIEQEVDWFYNSLGIDDLFFSRESPQ

SEQ ID NO: 39 - protein, 53 aa, Saccharomyces cerevisiae
KNSGTTKEIDELDSVRLQSKCEILEADNHSLEDKVNELEELVNSKFLDIENLN

SEQ ID NO: 40 - protein, 53 aa, Saccharomyces cerevisiae
NSSLLDNESLNENLFESQSMINPTSMEIQHPTLQLFENSSYSEYDQSDFEEDG

SEQ ID NO: 41 - protein, 53 aa, Arabidopsis thaliana
KAYQDELSAILGEKLSAEDEEEILAEFDNLESLLIVEDMPEVPTTELMPEEPE

SEQ ID NO: 42 - protein, 53 aa, Arabidopsis thaliana
NEEDWNLLEKLSMDGTEEFLKEALAFDNDESDAQDDANNEKEDDGEEFFQQIE

SEQ ID NO: 43 - protein, 53 aa, Arabidopsis thaliana
MEPPSSVVDDAEFWLPTEFLTDDDFLVEKENNSVGIDDSLFPYEPRHGFGTFG

SEQ ID NO: 44 - protein, 53 aa, Arabidopsis thaliana
NDNADLSIIFDSQDDFDNDITASIDFSSSIQFPASDQLQEQFDFTGIQLHQPP

SEQ ID NO: 45 - protein, 53 aa, Arabidopsis thaliana
KMVHNGIEYGDMQLISEAYDVLKNVGGLSNEELAEIFTEWNSGELESFLVEIT

SEQ ID NO: 46 - protein, 53 aa, Arabidopsis thaliana
FCINLTNEKLQQHFNQHVFKMEQDEYNKEEIDWSYIEFVDNQEILDLIEKKAG

SEQ ID NO: 47 - protein, 53 aa, Arabidopsis thaliana
ENDHTHLDSEGIQLIERNVEDYQELLDTNNNVLEDVSIGSILKEVSSYESFLE

SEQ ID NO: 48 - protein, 53 aa, Saccharomyces cerevisiae
YDYEQVQNADEELTFHENDVFDVFDDKDADWLLVKSTVSNEFGFIPGNYVEPE

SEQ ID NO: 49 - protein, 53 aa, Arabidopsis thaliana
ESVTLPQALNLDEFDLEDDTLDMEFDNHTRSEEDITLTDQIPTGIDPYVAVTF

SEQ ID NO: 50 - protein, 53 aa, Arabidopsis thaliana
EEKNRMIVPQETQTQPMFSEEDQSFWENLDVDDVFGLFNDDTNLEVPLQDHSS

SEQ ID NO: 51 - protein, 53 aa, Arabidopsis thaliana
REVPMFHCHDMSFKEEAPFTISDLSEENMLDSNYGDELSSEEFVLQDLQRASQ

SEQ ID NO: 52 - protein, 53 aa, Saccharomyces cerevisiae
LDYEFVGADLETAQTNFYWESVLNYTNSANISTTDTFENYHTYELDWHEDYVT

SEQ ID NO: 53 - protein, 53 aa, Arabidopsis thaliana
KTTKAQASEPEYFEEKRNLEDLWKATFSVGTEWDQQDALNEFNWDFTNLEEAL

SEQ ID NO: 54 - protein, 53 aa, Arabidopsis thaliana
YPEWSEFYLHNETEDEDEFMSPAFRESDCFILPENAEDKFITDNQFENSLGVY

SEQ ID NO: 55 - protein, 53 aa, Arabidopsis thaliana
EDQSFWENLDVDDVFGLFNDDTNLEVPLQDHSSTNEEDEFMIDISEYLSEEAM

SEQ ID NO: 56 - protein, 146 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTV

SEQ ID NO: 57 - protein, 64 aa, Saccharomyces cerevisiae
PETKQKRTAQNRAAQRAFRERKERKMKELEKKVQSLESIQQQNEVEATFLRDQLITLVNELKKY

SEQ ID NO: 58 - protein, 163 aa, Saccharomyces cerevisiae
PETKQKRTAQNRAAQRAFRERKERKMKELEKKVQSLESIQQQNEVEATFLRDQLITLVNELKKYSEQIDNOORKQDLDPETKQKRTAQNRAAQRAFRERKERKMKELEKKVQSLESIQQQNEVEATFLRDQLITLVNELKKYRPETRNDSKVLEYLARRDPNL

SEQ ID NO: 59 - protein, 54 aa, Saccharomyces cerevisiae
IFTNNLPFLNNNSINNNHSHNSSHNNNSPSIANNTNANTNTNTSASTNTNSPLL

SEQ ID NO: 60 - protein, 64 aa, Saccharomyces cerevisiae
DDHFIFTNNLPFLNNNSINNNHSHNSSHNNNSPSIANNTNANTNTNTSASTNTNSPLLRRNPSP

SEQ ID NO: 61 - protein, 61 aa, Saccharomyces cerevisiae
KKEKSPKGKSSISPQARAFLEQVFRRKQSLNSKEKEEVAKKCGITPLQVRVWFINKRMRSK

SEQ ID NO: 62 - protein, 68 aa, Saccharomyces cerevisiae
STKPYRGHRFTKENVRILESWFAKNIENPYLDTKGLENLMKNTSLSRIQIKNWVSNRRRKEKTITIAP

SEQ ID NO: 63 - protein, 55 aa, Saccharomyces cerevisiae
RRKIEIKFIENKTRRHVTFSKRKHGIMKKAFELSVLTGTQVLLLVVSETGLVYTF

SEQ ID NO: 64 - protein, 81 aa, Saccharomyces cerevisiae
KERRKIEIKFIENKTRRHVTFSKRKHGIMKKAFELSVLTGTQVLLLVVSETGLVYTFSTPKFEPIVTQQEGRNLIQACLNA

SEQ ID NO: 65 - protein, 8 aa, Saccharomyces cerevisiae
GXXIRXRF

SEQ ID NO: 66 - protein, 8 aa, Saccharomyces cerevisiae
GXSIRXRF

SEQ ID NO: 67 - protein, 10 aa, Saccharomyces cerevisiae
GNSIRHRFRV

SEQ ID NO: 68 - protein, 8 aa, Saccharomyces cerevisiae
GGAIRXRF

SEQ ID NO: 69 - protein, 7 aa, SV40 virus
PKKKRKV

SEQ ID NO: 70 - protein, 9 aa, Homo sapiens
PAAKRVKLD

SEQ ID NO: 71 - protein, 9 aa, Escherichia coli
KLKIKRPVK

SEQ ID NO: 72 - protein, 16 aa, Xenopus laevis
KRXXXXXXXXXXKKKK

SEQ ID NO: 73 - protein, 16 aa, Xenopus laevis
KRPAATKKAGQAKKKK

SEQ ID NO: 74 - protein, 20 aa, Xenopus laevis
AVKRPAATKKAGQAKKKKLD

SEQ ID NO: 75 - protein, 25 aa, Caenorhabditis elegans
MSRRRKANPTKLSENAKKLAKEVEN

SEQ ID NO: 76 - protein, 8 aa, Homo sapiens
YNDFGNYN

SEQ ID NO: 77 - protein, 5 aa, Homo sapiens
FGNYN

SEQ ID NO: 78 - protein, 7 aa, Homo sapiens
SNFGPMK

SEQ ID NO: 79 - protein, 4 aa, Homo sapiens
NFGG

SEQ ID NO: 80 - protein, 6 aa, Homo sapiens
GPYGGG

SEQ ID NO: 81 - protein, 42 aa, Homo sapiens
GNYNNQSSNFGPMKGGNFGGRSSGPYGGGGQYFAKPRNQGGY

SEQ ID NO: 82 - protein, 45 aa, Homo sapiens
FGNYNQQPSNYGPMKSGNFGGSRNMGGPYGGGNYGPGGSGGSGGY

SEQ ID NO: 83 - protein, 43 aa, Xenopus laevis
FGNYNSQSSSNFGPMKGGNYGGRNSGPYGGGYGGGSASSSSGY

SEQ ID NO: 84 - protein, 49 aa, Xenopus laevis
FGNYNQQSSNYGPMKSGGNFGGNRSMGGGPYGGGNYGPGNASGGNGGGY

SEQ ID NO: 85 - protein, 5 aa, Saccharomyces cerevisiae
KIPIK

SEQ ID NO: 86 - protein, 286 aa, Saccharomyces cerevisiae
MSDIEEGTPTNNGQQKERRKIEIKFIENKTRRHVTFSKRKHGIMKKAFELSVLTGTQVLLLVVSETGLVYTFSTPKFEPIVTQQEGRNLIQACLNAPDDEEEDEEEDGDDDDDDDDDGNDMQRQQPQQQQPQQQQQVLNAHANSLGHLNQDQVPAGALKQEVKSQLLGGANPNQNSMIQQQQHHTQNSQPQQQQQQQPQQQMSQQQMSQHPRPQQGIPHPQQSQPQQQQQQQQQLQQQQQQQQQQPLTGIHQPHQQAFANAASPYLNAEQNAAYQQYFQEPQQGQY

SEQ ID NO: 87 - protein, 126 aa, Saccharomyces cerevisiae
MDDICSMAENINRTLFNILGTEIDEINLNTNNLYNFIMESNLTKVEQHTLHKNISNNRLEIYHHIKKEKSPKGKSSISPQARAFLEQVFRRKQSLNSKEKEEVAKKCGITPLQVRVWFINKRMRSK

SEQ ID NO: 88 - protein, 210 aa, Saccharomyces cerevisiae
MNKIPIKDLLNPQITDEFKSSILDINKKLFSICCNLPKLPESVTTEEEVELRDILGFLSRANKNRKISDEEKKLLQTTSQLTTTITVLLKEMRSIENDRSNYQLTQKNKSADGLVFNVVTQDMINKSTKPYRGHRFTKENVRILESWFAKNIENPYLDTKGLENLMKNTSLSRIQIKNWVSNRRRKEKTITIAPELADLLSGEPLAKKKE

SEQ ID NO: 89 - protein, 650 aa, Saccharomyces cerevisiae
MSVSTAKRSLDVVSPGSLAEFEGSKSRHDEIENEHRRTGTRDGEDSEQPKKKGSKTSKKQDLDPETKQKRTAQNRAAQRAFRERKERKMKELEKKVQSLESIQQQNEVEATFLRDQLITLVNELKKYRPETRNDSKVLEYLARRDPNLHFSKNNVNHSNSEPIDTPNDDIQENVKQKMNFTFQYPLDNDNDNDNSKNVGKQLPSPNDPSHSAPMPINQTQKKLSDATDSSSATLDSLSNSNDVLNNTPNSSTSMDWLDNVIYTNRFVSGDDGSNSKTKNLDSNMFSNDFNFENQFDEQVSEFCSKMNQVCGTRQCPIPKKPISALDKEVFASSSILSSNSPALTNTWESHSNITDNTPANVIATDATKYENSFSGFGRLGFDMSANHYVVNDNSTGSTDSTGSTGNKNKKNNNNSDDVLPFISESPFDMNQVTNFFSPGSTGIGNNAASNTNPSLLQSSKEDIPFINANLAFPDDNSTNIQLQPFSESQSQNKFDYDMFFRDSSKEGNNLFGEFLEDDDDDKKAANMSDDESSLIKNQLINEEPELPKQYLQSVPGNESEISQKNGSSLQNADKINNGNDNDNDNDVVPSKEGSLLRCSEIWDRITTHPKYSDIDVDGLCSELMAKAKCSERGVVINAEDVQLALNKHMN

SEQ ID NO: 90 - protein, 510 aa, Saccharomyces cerevisiae
MHVFFPLLFRPSPVLFIACAYIYIDIYIHCTRCTVVNITMSTNRVPNLDPDLNLNKEIWDLYSSAQKILPDSNRILNLSWRLHNRTSFHRINRIMQHSNSIMDFSASPFASGVNAAGPGNNDLDDTDTDNQQFFLSDMNLNGSSVFENVFDDDDDDDDVETHSIVHSDLLNDMDSASQRASHNASGFPNFLDTSCSSSFDDHFIFTNNLPFLNNNSINNNHSHNSSHNNNSPSIANNTNANTNTNTSASTNTNSPLLRRNPSPSIVKPGSRRNSSVRKKKPALKKIKSSTSVQSSATPPSNTSSNPDIKCSNCTTSTTPLWRKDPKGLPLCNACGLFLKLHGVTRPLSLKTDIIKKRQRSSTKINNNITPPPSSSLNPGAAGKKKNYTASVAASKRKNSLNIVAPLKSQDIPIPKIASPSIPQYLRSNTRHHLSSSVPIEAETFSSFRPDMNMTMNMNLHNASTSSFNNEAFWKPLDSAIDHHSGDTNPNSNMNTTPNGNLSLDWLNLNL

SEQ ID NO: 91 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 92 - protein, 7 aa, Homo sapiens
SNYGPMK

SEQ ID NO: 93 - protein, 4 aa, Homo sapiens
NYGG

SEQ ID NO: 94 - protein, 8 aa, Saccharomyces cerevisiae
GPSIRXRF

SEQ ID NO: 95 - protein, 8 aa, Saccharomyces cerevisiae
GPAIRXRF

SEQ ID NO: 96 - protein, 8 aa, Saccharomyces cerevisiae
GASIRXRF

SEQ ID NO: 97 - protein, 8 aa, Saccharomyces cerevisiae
GAAIRXRF

SEQ ID NO: 98 - protein, 8 aa, Saccharomyces cerevisiae
GRSIRXRF

SEQ ID NO: 99 - protein, 8 aa, Saccharomyces cerevisiae
GRAIRXRF

SEQ ID NO: 100 - DNA, 24 bp, Synthetic construct
  source 1..24: mol_type = unassigned DNA
gcgggctctacttcatcggctagc

SEQ ID NO: 101 - DNA, 21 bp, Synthetic construct
  source 1..21: mol_type = unassigned DNA
tgataactagctgagggcccg

SEQ ID NO: 102 - DNA, 24 bp, Synthetic construct
  source 1..24: mol_type = unassigned DNA
gcgggctctacttcatcggctagc

SEQ ID NO: 103 - DNA, 59 bp, Synthetic construct
  source 1..59: mol_type = unassigned DNA
aattcgcttatttagaagtggcgcgccnnnnnnnnnnncgggccctcagctagttatca

SEQ ID NO: 104 - DNA, 24 bp, Synthetic construct
  source 1..24: mol_type = unassigned DNA
gcgggctctacttcatcggctagc

(19) United States
(12) Patent Application Publication
(10) Pub. No.: US 2025/0074948 A1
(43) Pub. Date: Mar. 6, 2025

(54) SYNTHETIC TRANSCRIPTION FACTORS

(71) Applicant: THE REGENTS OF THE UNIVERSITY OF CALIFORNIA, Oakland, CA (US)

(72) Inventors: Niklas F.C. HUMMEL, San Francisco, CA (US); Patrick M. SHIH, Berkeley, CA (US)

(21) Appl. No.: 18/821,489

(22) Filed: Aug. 30, 2024

Related U.S. Application Data
(60) Provisional application No. 63/579,836, filed on Aug. 31, 2023.

(51) Int. Cl. C07K 14/00; C12N 9/22

(52) U.S. Cl. CPC C07K 14/001; C12N 9/22; C07K 2319/09; C07K 2319/80

(57) ABSTRACT

The present invention provides for a synthetic transcription factor (TF) comprising (a) a DNA-binding domain of a transcription factor linked to (b) an activator domain (AD), and (c) optionally a nuclear localization sequence (NLS). The present invention provides for a nucleic acid encoding an activator domain (AD) of the present invention.

### CROSS REFERENCE TO RELATED APPLICATIONS

This application claims the priority benefit of U.S. Provisional Application Nos. 63/579,836, filed Aug. 31, 2023, which is hereby incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of synthetic transcription factors.

### REFERENCE TO SEQUENCE LISTING

The instant application contains a Sequence Listing which has been submitted electronically in XML format and is hereby incorporated by reference in its entirety. Said XML copy, created on Aug. 30, 2024, is named “2023-135-02 Sequence Listing.xml” and is 96 kilobytes in size.

### BACKGROUND OF THE INVENTION

Transcription factors (TFs) regulate gene expression by binding specific DNA regions with their DNA-binding domains (DBDs) and interacting with protein complexes through their transcriptional activator domains (TEDs) (2). TEDs that promote transcription are further classified as activation domains (ADs). New high-throughput methodologies have helped characterize the regulatory activity of transcriptional activator domains en masse in yeast, human, and fly models (3-9), and these approaches are beginning to be implemented to study plants (10). Still, most studies have biased their focus on chromatin regulators, TFs and coactivators, leaving other nuclear proteins and their potential role in transcription largely understudied. Furthermore, non-nuclear proteins can also be involved in transcriptional regulation, e.g. Notch1, a plasma membrane localized protein in multicellular animals, contains a C-terminal AD which gets localized to the nucleus during signal transduction and induces transcription (11). Similarly, the cytoplasmic beta-catenin is localized to the nucleus when multimerized where it acts as a transcriptional coactivator in fly and vertebrates and the closest plant homologs have been linked to root development (12-14). Thus, there is evidence of proteins with ADs outside of commonly studied transcriptional protein classes. Genome wide screens for putative TEDs can identify previously overlooked molecular factors that may play a role in transcriptional regulation.

The availability of large AD activity datasets has enabled the development of deep convolutional networks that can predict the activity of eukaryotic ADs from protein sequences (1, 6). These models have helped elucidate how specific amino acid sequence features of acidic ADs enable their transcriptional activation activity (15). Furthermore, Staller et al. show how acidic residues promote the exposure of hydrophobic residues that in turn are essential for AD activity (16). Hence, the distribution of acidic and hydrophobic residues is key, as hydrophobic clusters can lead to the intramolecular collapse of the AD, diminishing its activity (7). The recently proposed acidic exposure model links these observations to structural disorder in ADs, where acidic residues stabilize an energetically unfavorable solvent exposure of hydrophobic residues which in turn interact with coactivators to promote transcription in a transiently structured fashion (7). Thus, sequence composition, structural disorder and small sequence motifs in ADs have been linked to defining AD activity but we still lack full understanding of how positional sequence features affect AD function.

Eukaryotic transcription is facilitated by TFs, coactivators, and chromatin regulators. Coactivators can function as adaptors between TFs and RNA Polymerase II or the general transcription apparatus, while other coactivators modify chromatin to help transcription of chromatinized templates or help with unwinding DNA, all resulting in higher transcriptional output (11-14, 17-19). Coactivators interface between TFs and RNA polymerase but do not directly bind DNA, functionally separating them from TFs. Coactivators and chromatin regulators can contain ADs (1, 8, 20), marking activator activity non-unique to TFs. Still, there are currently no high-throughput methods for the annotation of new coactivator candidates, due to the multitude of mechanisms that coactivators use to promote transcription. Hence, the occurrence of ADs in nuclear non-TF genes could indicate that the underlying protein is involved in transcription and help annotate previously unknown transcription associated genes and coactivators. Importantly, because current AD predictive models have been trained on large datasets from select organisms (I.e., yeast and human), the predictive strength of these models in other eukaryotes has not been well defined.

The role of transcription factors in controlling gene regulation in plants has been studied to understand their ability to adapt to environmental changes. However, the role of transcriptional coactivators has been much less studied and leaves a large blind spot in our understanding of their role in transcription. Moreover, the complex physiology and cell wall of plants has hindered the implementation of high-throughput methods for the characterization of ADs in plants. As a result, our understanding of plant ADs and the role of potential coactivators pales in comparison to other better studied model eukaryotes (e.g. yeast, human, etc.). It was previously reported that a machine learning model trained on data from a large library of synthetic activators from yeast can correctly localize ADs in plant TFs (21); however, it is still unclear how applicable and scalable these models are in plant systems, necessitating further evaluation of more plant ADs predicted by yeast models. A larger set of validated plant ADs would allow the comparison of sequence features in plant ADs with observations in other well-studied eukaryotes. Moreover, studying ADs from non-TF genes can help us annotate previously unknown functions of the transcriptional machinery in plants unrestricted by evolutionary distance and deepen our understanding of the features defining AD strength in plants.

### SUMMARY OF THE INVENTION

The present invention provides for a synthetic transcription factor (TF) comprising (a) a DNA-binding domain of a transcription factor linked to (b) an activator domain (AD), and (c) optionally a nuclear localization sequence (NLS). The synthetic TF is a fusion protein comprising: (a) a DNA-binding domain of a transcription factor linked to (b) an activator domain (AD), and (c) optionally a nuclear localization sequence (NLS)

The present invention provides for a fusion protein or synthetic TF comprising the activator domain (AD) described herein linked to any other peptide, such a DNA-binding domain and/or a NLS, or any peptide heterologous to the AD, or the like. The present invention provides for a nucleic acid encoding the fusion protein or synthetic TF of the present invention.

In some embodiments, the DNA-binding domain is a DNA-binding domain of a eukaryotic TF or a prokaryotic TF. In some embodiments, the DNA-binding domain is a DNA-binding domain of a eukaryotic TF. In some embodiments, the DNA-binding domain is a deactivated RNA-guided nuclease variant of Cas9 (dCas9). In some embodiments, the DNA-binding domain is about 8, 10, 20, 30, 40, 50, 60, 70, 80, 90, 100, 110, 120, 130, 140, 146, or 150 amino acid residues long, or within a range of any two preceding values.

In some embodiments, the eukaryotic TF is a yeast TF. In some embodiments, the yeast TF is a Saccharomyces TF. In some embodiments, the Saccharomyces TF is a Saccharomyces cerevisiae TF.

In some embodiments, the S. cerevisiae TF is Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Matα2, Mga2, Met4, Mig1, Rap1, Rgt1, Rlm1, Smp1, Rme1, Rox1, Rtg3, Spt23, Tea1, Ume6, or Zap1. In some embodiments, the S. cerevisiae TF is Gal4, YAP1, GAT1, MATAL1, MATAL2, or MCM1.

In some embodiments, the S. cerevisiae TF is Gal4. In some embodiments, the DNA-binding domain comprises the amino acid sequence of Gal4 or MKLLSSIEQA CDICRLKKLK CSKEKPKCAK CLKNNWECRY SPKTKRSPLT RAHLTEVESR LERLEQLFLL IFPREDLDMI LKMDSLQDIK ALLTGLFVQD NVNKDAVTDR LASVETDMPL TLRQHRISAT SSSEESSNKG QRQLTV (SEQ ID NO:56).

In some embodiments, the S. cerevisiae TF is YAP1. In some embodiments, the DNA-binding domain comprises the amino acid sequence of YAP1. PETKQKR TAQNRAAQRA FRERKERKMK ELEKKVQSLE SIQQQNEVEA TFLRDQLITL VNELKKY (SEQ ID NO:57) or KQ DLDPETKQKR TAQNRAAQRA FRERKERKMK ELEKKVQSLE SIQQQNEVEA TFLRDQLITL VNELKKYRPE TRNDSKVLEY LARRDPNL (SEQ ID NO:58).

In some embodiments, the S. cerevisiae TF is GAT1. In some embodiments, the DNA-binding domain comprises the amino acid sequence of GAT1, IFTNNLP FLNNNSINNN HSHNSSHNNN SPSIANNTNA NTNTNTSAST NTNSPLL (SEQ ID NO:59) or D DHFIFTNNLP FLNNNSINNN HSHNSSHNNN SPSIANNTNA NTNTNTSAST NTNSPLLRRN PSP (SEQ ID NO:60).

In some embodiments, the S. cerevisiae TF is MATAL1. In some embodiments, the DNA-binding domain comprises the amino acid sequence of MATAL1 or KKEKS PKGKSSISPQ ARAFLEQVFR RKQSLNSKEK EEVAKKCGIT PLQVRVWFIN KRMRSK (SEQ ID NO:61).

In some embodiments, the S. cerevisiae TF is MATAL2. In some embodiments, the DNA-binding domain comprises the amino acid sequence of MATAL2 or STKP YRGHRFTKEN VRILESWFAK NIENPYLDTK GLENLMKNTS LSRIQIKNWV SNRRRKEKTI TIAP (SEQ ID NO:62).

In some embodiments, the S. cerevisiae TF is MCM1. In some embodiments, the DNA-binding domain comprises the amino acid sequence of MCM1, RRK IEIKFIENKT RRHVTFSKRK HGIMKKAFEL SVLTGTQVLL LVVSETGLVY TF (SEQ ID NO:63) or KERRK IEIKFIENKT RRHVTFSKRK HGIMKKAFEL SVLTGTQVLL LVVSETGLVY TFSTPKFEPI VTQQEGRNLI QACLNA (SEQ ID NO:64).

In some embodiments, the S. cerevisiae TF is Rap1. In some embodiments, the DNA-binding domain comprises the amino acid sequence of Rap1, or GXXIRXRF (wherein X is any amino acid) (SEQ ID NO:65), GGSIRXRF (wherein X is any amino acid) (SEQ ID NO:66), GGAIRXRF (wherein X is any amino acid) (SEQ ID NO:68), GPSIRXRF (wherein X is any amino acid) (SEQ ID NO:94), GPAIRXRF (wherein X is any amino acid) (SEQ ID NO:95), GASIRXRF (wherein X is any amino acid) (SEQ ID NO:96), GAAIRXRF (wherein X is any amino acid) (SEQ ID NO:97), GRSIRXRF (wherein X is any amino acid) (SEQ ID NO:98), GRAIRXRF (wherein X is any amino acid) (SEQ ID NO:99), or GNSIRHRFRV (SEQ ID NO:67).

In some embodiments, the activator domain comprises the amino acid sequence of one of SEQ ID NOs:1-55. In some embodiments, the activator domain has the capability to effect a “log 2_GFP foldchange” (using the conditions as described herein) of equal to or more than about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.00, or any value within any two preceding values. In some embodiments, the activator domain comprises an amino acid sequence having equal to or more than 70%, 75%, 80%, 85%, 90%, 95%, or 99% amino acid identity to any one of SEQ ID NOs:1-55, and optionally (a) comprises at least about one, two, three. four, five, six, seven, eight, nine, ten, 11, 12, 13, 14, 15, 16, 17, 18, 19, or 20, and/or equal to or more than 10%, 20%, 30%, 40%, 50%, 60%, 70%, 80%, 90%, or 100% of the acidic and/or hydrophobic amino acid residues, and/or comprises equal to or fewer basic amino acid residues, of the corresponding SEQ ID NOs:1-55.

In some embodiments, the acidic amino acid residue is Glu and/or Asp. In some embodiments, the hydrophobic amino acid residue is Ala, Val, Iso, Leu, Met, Phe, Tyr and/or Trp. In some embodiments, the basic amino acid residue is Arg, Lys and/or His.

In some embodiments, the NLS is monopartite. In some embodiments, the NLS comprises the amino acid sequence KKXK wherein X is any amino acid residue, KKXR wherein X is any amino acid residue, KRXK wherein X is any amino acid residue, KRXR wherein X is any amino acid residue, PKKKRKV (SV40 Large T-antigen) (SEQ ID NO:69), PAAKRVKLD (c-Myc) (SEQ ID NO:70) or KLKIKRPVK (TUS-protein) (SEQ ID NO:71).

In some embodiments, the NLS is bipartite. In some embodiments, the NLS comprises the amino acid sequence KRX₁₀KKKK (SEQ ID NO:72), KRPAATKKAGQAKKKK (SEQ ID NO:73) or AVKRPAATKKAGQAKKKKLD (nucleoplasmin NLS) (SEQ ID NO:74) or MSRRRKANPTKLSENAKKLAKEVEN (EGL-13) (SEQ ID NO:75).

In some embodiments, the NLS comprises a M9 domain or PY-NLS motif. In some embodiments, the NLS comprises the M9 domain comprising the amino acid sequence (a) one or more of YNDFGNYN (SEQ ID NO:76) or FGNYN (SEQ ID NO:77), SNFGPMK (SEQ ID NO:78), SNYGPMK (SEQ ID NO:92), NFGG (SEQ ID NO:79), NYGG (SEQ ID NO:93), GPYGGG (SEQ ID NO:80), (b) GNYNNQS SNFGPMKGGN FGGRSSGPYG GGGQYFAKPR NQGGY (hnRNP A1) (SEQ ID NO:81), (c) FGNYNQQPSN YGPMKSGNFG GSRNMGGPYG GGNYGPGGSG GSGGY(hnRNP A2/B1) (SEQ ID NO:82), (d) FGNYNSQSSS NFGPMKGGNY GGRNSGPYGG GYGGGSASSS SGY (Xenopus RNP A1) (SEQ ID NO:83), or (e) FGNYNQQSSN YGPMKSGGNF GGNRSMGGGP YGGGNYGPGN ASGGNGGGY (Xenopus RNP A2) (SEQ ID NO:84).

In some embodiments, the NLS comprises the amino acid sequence KIPIK (yeast Matα2) (SEQ ID NO:85). In some embodiments, the NLS is about 5, 10, 20, 30, 40, 50, 55, or 60 amino acid residues long, or within a range of any two preceding values.

In some embodiments, wherein any two, or all, of the DNA-binding domain, the activator domain, and the NLS are heterologous to each other.

In some embodiments, wherein one or more, or all, of the DNA-binding domain, the activator domain, and the NLS are obtained or derived from a non-viral organism.

In some embodiments, the DNA-binding domain, the NLS, and the activator domain are linked in this order from N- to C-terminus. Exemplary synthetic TF include, but are not limited to, the following:

The amino acid sequence of MCM1 is as follows:

(SEQ ID NO: 86)
MSDIEEGTPTNNGQQKERRKIEIKFIENKTRRHVTFSKRKHGIMKKAFE
LSVLTGTQVLLLVVSETGLVYTFSTPKFEPIVTQQEGRNLIQACLNAPD
DEEEDEEEDGDDDDDDDDDGNDMQRQQPQQQQPQQQQQVLNAHANSLGH
LNQDQVPAGALKQEVKSQLLGGANPNQNSMIQQQQHHTQNSQPQQQQQQ
QPQQQMSQQQMSQHPRPQQGIPHPQQSQPQQQQQQQQQLQQQQQQQQQQP
LTGIHQPHQQAFANAASPYLNAEQNAAYQQYFQEPQQGQY.

The amino acid sequence of MATAL1 is as follows:

(SEQ ID NO: 87)
MDDICSMAENINRTLFNILGTEIDEINLNTNNLYNFIMESNLTKVEQHT
LHKNISNNRLEIYHHIKKEKSPKGKSSISPQARAFLEQVFRRKQSLNSK
EKEEVAKKCGITPLQVRVWFINKRMRSK.

The amino acid sequence of MATAL2 is as follows:

(SEQ ID NO: 88)
MNKIPIKDLLNPQITDEFKSSILDINKKLFSICCNLPKLPESVTTEEEV
ELRDILGFLSRANKNRKISDEEKKLLQTTSQLTTTITVLLKEMRSIEND
RSNYQLTQKNKSADGLVFNVVTQDMINKSTKPYRGHRFTKENVRILESW
FAKNIENPYLDTKGLENLMKNTSLSRIQIKNWVSNRRRKEKTITIAPEL
ADLLSGEPLAKKKE.

The amino acid sequence of Yap1 is as follows:

(SEQ ID NO: 89)
MSVSTAKRSLDVVSPGSLAEFEGSKSRHDEIENEHRRTGTRDGEDSEQP
KKKGSKTSKKQDLDPETKQKRTAQNRAAQRAFRERKERKMKELEKKVQS
LESIQQQNEVEATFLRDQLITLVNELKKYRPETRNDSKVLEYLARRDPN
LHFSKNNVNHSNSEPIDTPNDDIQENVKQKMNFTFQYPLDNDNDNDNSK
NVGKQLPSPNDPSHSAPMPINQTQKKLSDATDSSSATLDSLSNSNDVLN
NTPNSSTSMDWLDNVIYTNRFVSGDDGSNSKTKNLDSNMFSNDFNFENQ
FDEQVSEFCSKMNQVCGTRQCPIPKKPISALDKEVFASSSILSSNSPAL
TNTWESHSNITDNTPANVIATDATKYENSFSGFGRLGFDMSANHYVVND
NSTGSTDSTGSTGNKNKKNNNNSDDVLPFISESPFDMNQVTNFFSPGST
GIGNNAASNTNPSLLQSSKEDIPFINANLAFPDDNSTNIQLQPFSESQS
QNKFDYDMFFRDSSKEGNNLFGEFLEDDDDDKKAANMSDDESSLIKNQL
INEEPELPKQYLQSVPGNESEISQKNGSSLQNADKINNGNDNDNDNDVV
PSKEGSLLRCSEIWDRITTHPKYSDIDVDGLCSELMAKAKCSERGVVIN
AEDVQLALNKHMN.

The amino acid sequence of Gat1 is as follows:

(SEQ ID NO: 90)
MHVFFPLLFRPSPVLFIACAYIYIDIYIHCTRCTVVNITMSTNRVPNLD
PDLNLNKEIWDLYSSAQKILPDSNRILNLSWRLHNRTSFHRINRIMQHS
NSIMDFSASPFASGVNAAGPGNNDLDDTDTDNQQFFLSDMNLNGSSVFE
NVFDDDDDDDDVETHSIVHSDLLNDMDSASQRASHNASGFPNFLDTSCS
SSFDDHFIFTNNLPFLNNNSINNNHSHNSSHNNNSPSIANNTNANTNTN
TSASTNTNSPLLRRNPSPSIVKPGSRRNSSVRKKKPALKKIKSSTSVQS
SATPPSNTSSNPDIKCSNCTTSTTPLWRKDPKGLPLCNACGLFLKLHGV
TRPLSLKTDIIKKRQRSSTKINNNITPPPSSSLNPGAAGKKKNYTASVA
ASKRKNSLNIVAPLKSQDIPIPKIASPSIPQYLRSNTRHHLSSSVPIEA
ETFSSFRPDMNMTMNMNLHNASTSSFNNEAFWKPLDSAIDHHSGDTNPN
SNMNTTPNGNLSLDWLNLNL.

The present invention also provides for a nucleic acid encoding any one of the synthetic TF of the present invention operatively linked to a promoter capable of expressing the synthetic TF in vitro or in vivo.

The present invention provides for a nucleic acid encoding an activator domain of the present invention. In some embodiments, the activator domain comprises an amino acid sequence of SEQ ID NO:1-55. In some embodiments, the activator domain is about 50, 51, 52, 53, 54, or 55 amino acid residues long, or within a range of any two preceding values. In some embodiments, the activator domain comprises acidic amino acid residues (such as D and E) in one or more positions within positions 33 to 46. In some embodiments, the activator domain comprises disorder promoting amino acid residues (such as A, E, G, K, Q, S, and P) in one or more positions within positions 1 to 5. In some embodiments, the activator domain comprises hydrophobic amino acid residues (such as W, L, F, and Y) in one or more positions within positions 27 to 40, or positions 30 to 36. In some embodiments, the activator domain comprises order promoting amino acid residues (such as C, F, H, I, L, M, N, V, W, and Y) in one or more positions within positions 15 to 43, positions 18 to 34, positions 21 to 33, or positions 25, 29, 30, 31, or 32.

In some embodiments, the synthetic transcription factor (TF) comprises: (a) a DNA-binding domain of a transcription factor linked to (b) an activator domain (AD) comprising 50 to 55 amino acid residues, and having (i) one or more acidic amino acid residues in one or more positions within positions 33 to 46; (ii) one or more disorder promoting amino acid residues in one or more positions within positions 1 to 5; (iii) one or more hydrophobic amino acid residues in one or more positions within positions 27 to 40, or positions 30 to 36; and (iv) one or more order promoting amino acid residues in one or more positions within positions 15 to 43, positions 18 to 34, positions 21 to 33, or positions 25, 29, 30, 31, or 32. In some embodiments, each of the acidic amino acid residue is independently D or E. In some embodiments, each of the disorder promoting amino acid residue is independently A, E, G, K, Q, S, or P. In some embodiments, each of the hydrophobic amino acid residue is independently W, L, F, or Y. In some embodiments, each of the order promoting amino acid residue is independently C, F, H, I, L, M, N, V, W, or Y.

The present invention also provides for a vector comprising the nucleic acid of the present invention. In some embodiments, the vector is capable of stably integrating into a chromosome of a host cell or stably residing in a host cell. In some embodiments, the vector is an expression vector.

The present invention also provides for a host cell comprising the vector of the present invention, wherein the host cell is capable of expressing the synthetic TF or activator domain.

The present invention also provides for a system comprising a nucleic acid of the present invention and a second nucleic acid, or the nucleic acid, encodes a gene of interest (GOI) operatively linked to a promoter and one or more activator binding domains, or combination thereof, wherein the synthetic TF binds at least one of the one or more activator binding domain such that the synthetic TF modulates the expression of the GOI.

The present invention also provides for a genetically modified eukaryotic cell or organism, such as a plant cell or plant, comprising: (a) one or more nucleic acids each encoding one or more transcription activators operatively linked to a first promoter; and (b) one or more nucleic acids each encoding one or more independent genes of interest (GOI) each operatively linked to a promoter that is activated by the one or more transcription activators; wherein at least one transcription activator is a synthetic transcription factor (TF) of the present invention

In some embodiments, the first promoter, the second promoter, or both, is a tissue-specific or inducible promoter.

In some embodiments, any domain of the synthetic TF is heterologous to the plant cell or plant, one or more of the GOI, any other transcription activator, and/or any of the promoters.

In some embodiments, the transcription activator is heterologous to the eukaryotic cell or organism, such as a plant cell or plant, one or more of the GOI, any other or transcription activator, and/or any of the promoters.

In some embodiments, the genetically modified eukaryotic cell or organism, such as a plant cell or plant comprises: (a) a first nucleic acid encoding a transcription activator operatively linked to a first tissue-specific or inducible promoter, and (b) one or more nucleic acids each encoding one or more independent genes of interest (GOI) each operatively linked to a promoter that is activated by the transcription activators.

In some embodiments, the genetically modified eukaryotic cell or organism, such as a plant cell or plant comprises: (a) optionally a first nucleic acid encoding a transcription activator operatively linked to a first tissue-specific or inducible promoter; and (b) one or more nucleic acids each encoding one or more independent genes of interest (GOI) each operatively linked to a promoter that is activated by the transcription activators.

In some embodiments, the promoter is a tissue-specific promoter. Examples of tissue-specific promoters under developmental control include promoters that initiate transcription only (or primarily only) in certain tissues, such as vegetative tissues, cell walls, including e.g., roots or leaves. A variety of promoters specifically active in vegetative tissues, such as leaves, stems, roots and tubers are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarn promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are knI-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

In some embodiments, the promoter is substantially identical to the native promoter of a promoter that drives expression of a gene involved in secondary wall deposition. Examples of such promoters are promoters from IRX1, IRX3, IRX5, IRX8, IRX9, IRX14, IRX7, IRX10, GAUT13, or GAUT14 genes. Specific expression in fiber cells can be accomplished by using a promoter such as the NST1 promoter and specific expression in vessels can be accomplished by using a promoter such as VND6 or VND7. (See, e.g., PCT/US2012/023182 for illustrative promoter sequences). In some embodiments, the promoter is a secondary cell wall-specific promoter or a fiber cell-specific promoter. In some embodiments, the promoter is from a gene that is co-expressed in the lignin biosynthesis pathway (phenylpropanoid pathway). In some embodiments, the promoter is a C4H, C3H, HCT, CCR1, CAD4, CAD5, F5H, PAL1, PAL2, 4CL1, or CCoAMT promoter. In some embodiments, the tissue-specific secondary wall promoter is an IRX1, IRX3, IRX5, IRX8, IRX9, IRX14, IRX7, IRX10, GAUT13, GAUT14, or CESA4 promoter. Suitable tissue-specific secondary wall promoters, and other transcription factors, promoters, regulatory systems, and the like, suitable for this present invention are taught in U.S. Patent Application Pub. Nos. 2014/0298539, 2015/0051376, and 2016/0017355.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In some embodiments, each GOI is operatively linked to a promoter that is activated by the transcription activator.

In some embodiments, the promoter comprises one or more DNA-binding sites specific for the transcription activator.

In some embodiments, the promoter comprises 1, 2, 3, 4, 5, 6, 7, 8, 9, or 10 DNA-binding sites specific for the transcription activator).

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Proteome-wide characterization of putative ADs mined from non-TF plant and yeast proteins. Histogram showing the PADDLE predicted activity of all 53 amino acid tiles in the (A) S. cerevisiae and (B) A. thaliana proteome. Inlet figures show the magnified areas of the histogram the putative AD candidates for the libraries were chosen from (marked in red). (C) The 12,000 strongest A. thaliana and 6,000 strongest S. cerevisiae tiles were characterized as a synthetic TF library in S. cerevisiae. Activator activity was calculated by abundance of barcodes in bins established during FACS sorting. DBD: DNA binding domain, EBD: estrogen binding domain. (D) Activity of every tile as determined by FACS and consecutive barcode sequencing across eight bins. Red bar indicates no-AD control. Predicted activity vs. experimentally observed activity of all tiles from (E) S. cerevisiae and (F) A. thaliana. SP: Spearman's R.

FIG. 2. Tiling ADs with single amino acid resolution deciphers positional distribution of key amino acid groups. Density of functional amino acids across every position of every tile in the quartile with the strongest and weakest activity (4388 sequences per quartile). Density is calculated in a five amino acid window for each position along the AD as the average of all (A) hydrophobic residues (W, L, F, Y), (B) acidic residues (D, E). Error bars indicate the 95% confidence interval. (C) Mean disorder at every position of tiles in respective quartiles predicted by Metapredict2. The red line indicates threshold for residues to be predicted as disordered with >0.5 indicating disorder. Error bars indicate the 95% confidence interval. (D) Example of PADDLE predicted vs measured activity in single amino acid resolution of regions of interest for the gene with the strongest derived tile (AT1G50780). Tiling protein sequences with single amino acid resolution allows us to observe the effects on activity when (E) gaining a C-terminal amino acid or (F) losing an N-terminal amino acid. Blue colored boxes indicate amino acids decreasing activity, red boxes indicate amino acids increasing activity.

FIG. 3. Non-nuclear proteins occurring throughout organelles have predicted ADs in both yeast and plants. Cell schematics with occurrence of parent genes with ADs in (A) S. cerevisiae and (B) A. thaliana. GO terms associated with nuclear genes in (C) S. cerevisiae and (D) A. thaliana. GO terms associated with non-nuclear genes in (C) S. cerevisiae and (D) A. thaliana.

FIG. 4. Cross-validation of strong ADs yields candidates with similar activities to gold standard activators. (A) Activity of 55 of the 100 strongest tiles measured in yeast in N. benthamiana. VP16 and VPR serve as positive AD controls. EV: Empty Vector control, Gal4: Gal4-DBD without any AD. (B) Mean normalized in plant activity of ADs in (A) sorted by activity strength in yeast. (C) Mean normalized in plant activity of ADs in (A) relative to PADDLE predicted activity.

FIG. 5. TFs in both Arabidopsis and yeast show similar predicted AD strength as non-TF genes. PADDLE predicted activation of every 53 amino acid tiles from all annotated (A) A. thaliana and (B) S. cerevisiae TFs. Inlets show the same area of predicted activity as used for choosing tiles for the library in FIG. 1, panels A and B.

FIG. 6. Flow cytometry of library against GAL4 and VP16 controls. Flow Cytometry comparing 100,000 observed events of the uninduced/induced library vs. (A) Zif268-GCN4-AD and (B) Zif268-VP16-AD.

FIG. 7. Activity of tiles is highly reproducible. (A) Activities calculated from two independent sorts are strongly positively correlated. PADDLE predictions are monotonically correlated with experimental observations for combined Arabidopsis and yeast data.

FIG. 8. AD populations with similar amino acid composition show disparate amino acid residue distribution. (A) Amino acid frequencies in every AD quartile population. Quartile 4 contains the strongest ADs. Density of functional amino acids across every position of every tile in the quartile with the strongest and weakest activity (4388 sequences per quartile). Density is calculated in a five amino acid window for each position along the AD as the average of all (B) hydrophobic residues (W, L, F, Y), (C) acidic residues (D, E), (D) disorder promoting residues (A, E, G, K, Q, S, P) and (E) order promoting residues (C, F, H, I, L, M, N, V, W, Y) at the respective position. Error bars indicate the 95% confidence interval.

FIG. 9. Density of single amino acids varies along tiles between quartiles. Amino acid frequencies in every AD quartile population. Quartile 4 contains the strongest ADs. Density of functional amino acids across every position of every tile in the quartile with the strongest and weakest activity (4388 sequences per quartile). Density is calculated in a five amino acid window for each position along the AD as the average of the respective tile.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A “constitutive promoter” is one that is capable of initiating transcription in nearly all cell types, whereas a “cell type-specific promoter” initiates transcription only in one or a few particular cell types or groups of cells forming a tissue. In some embodiments, the promoter is secondary cell wall-specific and/or fiber cell-specific. A “fiber cell-specific promoter” refers to a promoter that initiates substantially higher levels of transcription in fiber cells as compared to other non-fiber cells of the plant. A “secondary cell wall-specific promoter” refers to a promoter that initiates substantially higher levels of transcription in cell types that have secondary cell walls, e.g., lignified tissues such as vessels and fibers, which may be found in wood and bark cells of a tree, as well as other parts of plants such as the leaf stalk. In some embodiments, a promoter is fiber cell-specific or secondary cell wall-specific if the transcription levels initiated by the promoter in fiber cells or secondary cell walls, respectively, are at least 3-fold, 4-fold, 5-fold, 6-fold, 7-fold, 8-fold, 9-fold, 10-fold, 50-fold, 100-fold, 500-fold, 000-fold higher or more as compared to the transcription levels initiated by the promoter in other tissues, resulting in the encoded protein substantially localized in plant cells that possess fiber cells or secondary cell wall, e.g., the stem of a plant. Non-limiting examples of fiber cell and/or secondary cell wall specific promoters include the promoters directing expression of the genes IRX1, IRX3, IRX5, IRX7, IRX8, IRX9, IRX10, IRX14, NST1, NST2, NST3, MYB46, MYB58, MYB63, MYB83, MYB85, MYB103, PAL1, PAL2, C3H, CcOAMT, CCR1, F5H, LAC4, LAC17, CADc, and CADd. See, e.g., Turner et al 1997; Meyer et al 1998; Jones et al 2001; Franke et al 2002; Ha et al 2002; Rohde et al 2004; Chen et al 2005; Stobout et al 2005; Brown et al 2005; Mitsuda et al 2005; Zhong et al 2006; Mitsuda et al 2007; Zhong et al 2007a, 2007b; Zhou et al 2009; Brown et al 2009; McCarthy et al 2009; Ko et al 2009; Wu et al 2010; Berthet et al 2011. In some embodiments, a promoter is substantially identical to a promoter from the lignin biosynthesis pathway. A promoter originated from one plant species may be used to direct gene expression in another plant species.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The present invention provides for a toolbox or library of strong plant transcriptional activators that enable us strong upregulation of gene expression in plants. The library enables us to modulate transcription specifically and is easy to implement into different expression systems as well as fusion proteins.

In some embodiments, the toolbox or library of plant transcription factor based regulatory domains that enable strong enhancement of gene expression in plants. The parts work by being tethering to a DNA binding domain of any one of interest and allow strong activation at any locus the transcription factor can be targeted to.

The present invention provides for a method for fast throughput characterization of plant regulatory domains while excluding native DNA binding activity. The method comprises: scanning a library of transcription factors, such as plant transcription factors, such as Arabidopsis thaliana transcription factors, for their DNA binding domains; generating a truncation library excluding the native DNA binding activity or native DNA binding domain; and characterizing of the regulatory domains of the transcription factors. In some embodiments, the characterizing step is parallel to the other steps.

The present invention can be useful for: controlling gene expression in plants; inclusion in a known or novel expression systems, such as for increasing yields in protein expression using this technology.

In some embodiments, the synthetic TF of the present invention do not contain any viral or mammalian parts, or nucleic acid sequence of a viral or mammalian origin.

The synthetic TF of the present invention can be used in the invention taught in PCT International Patent Application No. PCT/US2018/050514 (Publication No. WO 2019/051503 A2), which is hereby incorporated by reference.

The present invention can be used in new or non-model organisms for the controlled expression of multiple genes in a certain manner, including expressing multiple genes simultaneously. The expression of these genes can be regulated in a temporal and/or spatial manner.

The present invention can be used in a strategy to design system utilizing synthetic promoters for the ultimate purpose of controlling expression strength, tissue-specificity, and environmentally-responsive promoters and associated downstream products (e.g. RNA, protein). This method utilizes the synthetic TF of the present invention with its corresponding DNA binding sequence (cis-element), where multiple slightly varying nucleotide sequences of cis-elements are concatenated to provide variability in the binding strength of the transcriptional regulator. The cis-elements are fused to varying minimal promoter sequences (minimal promoter or minimal promoter+UTR upstream sequence of ATG) of the eukaryote host organism of interest to enable the synthetic TF the ability to control expression of the target downstream gene. This invention provides a strategy for engineering an entirely orthogonal transcriptional network into any eukaryotic host for controlling expression strengths of multiple genes through the heterologous expression of the synthetic TF.

The present invention enables one skilled in the art to control the expression of a single or multiple genes simultaneously in any eukaryote organism with only one endogenous promoter using the synthetic TF. Many times, such as in plants, reuse of the same promoter to drive heterologous expression of multiple genes may increase the likelihood of gene silencing and even creates genome instability. Moreover, use of one endogenous promoter may offer the desired expression level required to express a gene of interest. The present invention offers the capacity of retaining expression specificity while offering a dynamic range of expression of the transgene using the synthetic TF. For example, there are many promoters that display tissue-specific expression in one specific tissue (e.g., plant roots, seeds, leaves, or the like). By utilizing a promoter of interest to drive expression of the synthetic TF, one can generate a library of synthetic promoters that are turned on by the synthetic TF at varying expression strengths. This is an efficient and productive way in controlling the exact expression strength of a single or multiple genes in a tissue-specific or environmentally-responsive manner.

The present invention can be applied to any host eukaryotic organism of interest, such as fungi, plant, and animal cells, using the synthetic TF. This invention offers the ability to perform various permutations and test multiple expression profiles. For example, one set of plants could be generated with different promoters driving the synthetic TF (set A) and another set of plants would be transformed with different combination of synthetic promoters driving one or a multiple transgene of interests (set B). Plants from set A could be crossed with those of set B, this would great a 2D matrix of new plants expressing transgene of interests in different tissues and at different strength. This approach has the capacity to reduce number of transformations. For example, generation of 50 plants for each set (A and B) will require 100 transformations and will be used to generate 2500 combinations that would normally require 2500 independent transformations without the use of matrix as presented above. Such matrix approach is applicable to any eukaryotic host that can be crossed such as crops and yeast.

This invention can be used by nearly any biotechnology industry. This invention can easily be utilized for any eukaryotic host, such as plant, yeast or animal hosts.

The present invention provides for the following embodiments of the invention:

TABLE 1
Activator Domains (AD)

| SEQ | | | |
|---|---|---|---|
| ID | | | Real |
| NO | Amino Acid Sequence | Locus | ID |
| 1 | GDDKDSDDLNLFLNAIGEAGDEEGPTSFND | AT4G25330.1 | 95 |
| | IDFLTFDDEDLHNPFQDCETSPI | | |
| 2 | MMSDVDIFGNGGITFDDFLYIMAQNTSQES | AT5G42880.1 | 94 |
| | ASDELIEVFRVFDRDGDGLISQL | | |
| 3 | TSNQSGATSPTVFSGEFADDVDWSDENWPE | AT3G32030.1 | 93 |
| | LEFRSAEDEAWYAVEFSDICDAL | | |
| 4 | ASDMSGMVMDTSVLDSAFTSDVGPDGEGAG | AT1G70750.1 | 92 |
| | NSRDSLRSFDQIPWNFSLSDLTA | | |
| 5 | PKNLEFYIDEEDCHLIPVEFYKPSEEVREI | AT3G61700.2 | 91 |
| | SDINGDFILDFGVEHDFTAAAET | | |
| 6 | ALSKWASEGYMPTFDEYMEVGEVTGGMDDF | AT3G51920.1 | 90 |
| | ALYSFIAMEDCDEKPLYEWFDSK | | |
| 7 | DGERNSNVRESAQGKALMTSEQNSNRYWNS | YGR221C | 89 |
| | FHDEDDWNLFNGMELESNGVVTF | | |
| 8 | CPSQDVVIHDCGEIPEGADDGICDFFKDGD | AT2G19390.1 | 88 |
| | VYPDWPIDLNESPAELSWWMETV | | |
| 9 | EPSMQDFDPNFEGDLYYLPKMDSSMNSANS | YDR260C | 86 |
| | DSNATEKRFIYGGYDDFLQPSIE | | |
| 10 | CANIPEFDSFYENENINYNLESFAPLNCDV | YKL135C | 84 |
| | NSPFLPINNNDINVNAYGDENLT | | |
| 11 | VDIFGNGGITFDDFLYIMAQNTSQESASDE | YBL029W | 83 |
| | LIEVFRVFDRDGDGLISQLELGE | | |
| 12 | NEISSKANDDVLLDFDERDDVTNTNAGMLN | AT3G51920.1 | 82 |
| | TLTTLGDLDDLFDFGPSEDATQI | | |
| 13 | VNEDLQFDGFGADVESEFSVLNHMMEFNGY | YGR116W | 80 |
| | RSDRLEFDELEDDVSVIPLKGVN | | |
| 14 | RFYAELHIDDPIVTEYFKNQNTASIAELNS | AT3G51920.1 | 79 |
| | LQDIYDYLEFKYANEINEMFINH | | |
| 15 | IPDAENDLSFFDNGDKEKNDLFYGWGDIGN | AT3G51920.1 | 75 |
| | FEDVDNMLRSCDSTFGLDSLNNE | | |
| 16 | TSDVDGDGFIDFEEFLKLMEGEDGSDEERR | YHL023C | 74 |
| | KELKEAFGMYVMEGEEFITAASL | | |
| 17 | LSAEDEEEILAEFDNLESLLIVEDMPEVPT | AT2G15790.1 | 72 |
| | TELMPEEPEKMDLPDVPTKAPVA | | |
| 18 | HTQYDVEEEDMEVSAMLQDGKISMNEIFFE | YEL040W | 68 |
| | EENFQDINKILEFDNDFVAEFCS | | |
| 19 | DESEPLDLSHLQIPDGLGGPDDFDTQAGDL | YPR160W | 66 |
| | SSWLNIDDDALPDTDDLLGLQIP | | |
| 20 | IWGFEDHVSNYGGLDFGSGVGDGGDYVAVE | AT1G13360.1 | 64 |
| | GLFEFSDDCFDSGDLFSWRSESL | | |
| 21 | LKEDTGSTLYEFAMKLEDLNEPLSPWISSA | YPL159C | 62 |
| | TGLEFFSEWENIPSELLKNLKPF | | |
| 22 | TSSAVTLPPTEEIDPMQGLSMDDEMKDVGF | YKR035W-A | 59 |
| | LPPIVSLDEFMESLNSEPPFGSP | | |
| 23 | LEDDTLDMEFDNHTRSEEDITLTDQIPTGI | YBL007C | 56 |
| | DPYVAVTFDEDIISESIPMDVDQ | | |
| 24 | QDGERNSNVRESAQGKALMTSEQNSNRYWN | YDR260C | 53 |
| | SFHDEDDWNLFNGMELESNGVVT | | |
| 25 | MSDVDIFGNGGITFDDFLYIMAQNTSQESA | AT3G51920.1 | 51 |
| | SDELIEVFRVFDRDGDGLISQLE | | |
| 26 | ISWDEFAEAIRAFSPSITSEEIDNMFREID | AT2G29920.1 | 45 |
| | VDGDNQIDVAEYASCLMLGGEGN | | |
| 27 | REVEEVVKTSDVDGDGFIDFEEFLKLMEGE | AT2G29920.1 | 44 |
| | DGSDEERRKELKEAFGMYVMEGE | | |
| 28 | LGIGWTSTMLSYERASWTDEFLNTSPSPEV | YGR004W | 43 |
| | FTLPEEQSGMAWEWHDKDWMLDL | | |
| 29 | DEIDMDDLVHFSPSIEFADTQLKSSGDFQL | AT1G50780.1 | 39 |
| | DDSWSSKDHEIFHFDPVTEFSDA | | |
| 30 | TFFLDDALSVLNSDKNSHLLSAVKRDFKDE | YGR221C | 37 |
| | DKVSLDEAIDLAWTNDEFDCLVD | | |
| 31 | MQDFDPNFEGDLYYLPKMDSSMNSANSDSN | AT5G42380.1 | 36 |
| | ATEKRFIYGGYDDFLQPSIENSQ | | |
| 32 | SGEELQSCVSLLGGALSSREVEEVVKTSDV | AT1G06670.1 | 35 |
| | DGDGFIDFEEFLKLMEGEDGSDE | | |
| 33 | VEMKWDFDSLSSSDYIIENNINLDALAEDN | YHR102W | 32 |
| | NEWATAQHDLFNYAYPDEDSYYF | | |
| 34 | DNQEIDFLDLFSPTFKFNFASTTTDWQPIV | AT2G18090.1 | 31 |
| | AGPDECDESVSDLLAEVEAMESQ | | |
| 35 | EELQSCVSLLGGALSSREVEEVVKTSDVDG | AT5G42380.1 | 30 |
| | DGFIDFEEFLKLMEGEDGSDEER | | |
| 36 | ENVVFITSTAGQGEFPQDGKSFWEALKNDT | YJR137C | 28 |
| | DLDLASLNVAVFGLGDSEYWPRK | | |
| 37 | YFEEKRSLEDLWKVAFPVGTEWDQLDALYE | YMR196W | 25 |
| | FNWDFQNLEEALEEGGKLYGKKV | | |
| 38 | VFDFPGKDLQREEVIDLLDQQGFIPDDLIE | YDL215C | 24 |
| | QEVDWFYNSLGIDDLFFSRESPQ | | |
| 39 | KNSGTTKEIDELDSVRLQSKCEILEADNHS | YHR158C | 23 |
| | LEDKVNELEELVNSKFLDIENLN | | |
| 40 | NSSLLDNESLNENLFESQSMINPTSMEIQH | YIR033W | 22 |
| | PTLQLFENSSYSEYDQSDFEEDG | | |
| 41 | KAYQDELSAILGEKLSAEDEEEILAEFDNL | AT5G09260.1 | 21 |
| | ESLLIVEDMPEVPTTELMPEEPE | | |
| 42 | NEEDWNLLEKLSMDGTEEFLKEALAFDNDE | AT1G50780.1 | 20 |
| | SDAQDDANNEKEDDGEEFFQQIE | | |
| 43 | MEPPSSVVDDAEFWLPTEFLTDDDFLVEKE | AT3G54000.1 | 18 |
| | NNSVGIDDSLFPYEPRHGFGTFG | | |
| 44 | NDNADLSIIFDSQDDFDNDITASIDFSSSI | AT1G04500.1 | 15 |
| | QFPASDQLQEQFDFTGIQLHQPP | | |
| 45 | KMVHNGIEYGDMQLISEAYDVLKNVGGLSN | AT1G64190.1 | 14 |
| | EELAEIFTEWNSGELESFLVEIT | | |
| 46 | FCINLTNEKLQQHFNQHVFKMEQDEYNKEE | AT1G06510.1 | 12 |
| | IDWSYIEFVDNQEILDLIEKKAG | | |
| 47 | ENDHTHLDSEGIQLIERNVEDYQELLDTNN | AT1G06510.1 | 10 |
| | NVLEDVSIGSILKEVSSYESFLE | | |
| 48 | YDYEQVQNADEELTFHENDVFDVFDDKDAD | YBL007C | 9 |
| | WLLVKSTVSNEFGFIPGNYVEPE | | |
| 49 | ESVTLPQALNLDEFDLEDDTLDMEFDNHTR | AT3G59550.1 | 8 |
| | SEEDITLTDQIPTGIDPYVAVTF | | |
| 50 | EEKNRMIVPQETQTQPMFSEEDQSFWENLD | AT1G50780.1 | 6 |
| | VDDVFGLFNDDTNLEVPLQDHSS | | |
| 51 | REVPMFHCHDMSFKEEAPFTISDLSEENML | AT3G12320.1 | 5 |
| | DSNYGDELSSEEFVLQDLQRASQ | | |
| 52 | LDYEFVGADLETAQTNFYWESVLNYTNSAN | YEL040W | 4 |
| | ISTTDTFENYHTYELDWHEDYVT | | |
| 53 | KTTKAQASEPEYFEEKRNLEDLWKATFSVG | AT5G64910.2 | 3 |
| | TEWDQQDALNEFNWDFTNLEEAL | | |
| 54 | YPEWSEFYLHNETEDEDEFMSPAFRESDCF | AT2G40120.1 | 2 |
| | ILPENAEDKFITDNQFENSLGVY | | |
| 55 | EDQSFWENLDVDDVFGLFNDDTNLEVPLQD | AT1G50780.1 | 1 |
| | HSSTNEEDEFMIDISEYLSEEAM | | |

A synthetic transcription factor (TF) comprising (a) a DNA-binding domain of a transcription factor linked to (b) an activator domain, and (c) a nuclear localization sequence (NLS).

In some embodiments, the DNA-binding domain is a DNA-binding domain of a eukaryotic TF or a prokaryotic TF.

In some embodiments, the DNA-binding domain is a DNA-binding domain of a eukaryotic TF.

In some embodiments, the eukaryotic TF is a yeast TF. In some embodiments, the yeast TF is a Saccharomyces TF. In some embodiments, the Saccharomyces TF is a Saccharomyces cerevisiae TF. In some embodiments, the S. cerevisiae TF is Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Matα2, Mga2, Met4, Mig1, Rap1, Rgt1, Rlm1, Smp1, Rme1, Rox1, Rtg3, Spt23, Tea1, Ume6, or Zap1. In some embodiments, the S. cerevisiae TF is Gal4, YAP1, GAT1, MATAL1, MATAL2, MCM1, or Rap1.

In some embodiments, the synthetic TF comprises the activator domain which is a herpes simplex virus VP16, maize C1, or a yeast activator domain.

In some embodiments, the activator domain is the yeast activator domain. In some embodiments, the yeast activator domain is a Saccharomyces activator domain. In some embodiments, the Saccharomyces activator domain is a Saccharomyces cerevisiae activator domain.

In some embodiments, the S. cerevisiae activator domain is a Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mga2, Met4, Rap1, Rlm1, Smp1, Rtg3, Spt23, Tea1, Ume6, or Zap1 activator domain.

In some embodiments, the NLS is monopartite or bipartite. In some embodiments, the NLS comprises a M9 domain or PY-NLS motif. In some embodiments, the NLS comprises the amino acid sequence KIPIK (yeast Matα2).

In some embodiments, any two, or all, of the DNA-binding domain, the activator domain, and the NLS are heterologous to each other.

In some embodiments, the dCas9 comprises the following amino acid sequence:

```
(SEQ ID NO: 91)
                10 20 30 40 50
MDKKYSIGLA IGTNSVGWAV ITDEYKVPSK KFKVLGNTDR HSIKKNLIGA
                60 70 80 90 100
LLFDSGETAE ATRLKRTARR RYTRRKNRIC YLQEIFSNEM AKVDDSFFHR
              110 120 130 140 150
LEESFLVEED KKHERHPIFG NIVDEVAYHE KYPTIYHLRK KLVDSTDKAD
              160 170 180 190 200
LRLIYLALAH MIKFRGHFLI EGDLNPDNSD VDKLFIQLVQ TYNQLFEENP
              210 220 230 240 250
INASGVDAKA ILSARLSKSR RLENLIAQLP GEKKNGLFGN LIALSLGLTP
              260 270 280 290 300
NFKSNFDLAE DAKLQLSKDT YDDDLDNLLA QIGDQYADLF LAAKNLSDAI
              310 320 330 340 350
LLSDILRVNT EITKAPLSAS MIKRYDEHHQ DLTLLKALVR QQLPEKYKEI
              360 370 380 390 400
FFDQSKNGYA GYIDGGASQE EFYKFIKPIL EKMDGTEELL VKLNREDLLR
              410 420 430 440 450
KQRTFDNGSI PHQIHLGELH AILRRQEDFY PFLKDNREKI EKILTFRIPY
              460 470 480 490 500
YVGPLARGNS RFAWMTRKSE ETITPWNFEE VVDKGASAQS FIERMTNFDK
              510 520 530 540 550
NLPNEKVLPK HSLLYEYFTV YNELTKVKYV TEGMRKPAFL SGEQKKAIVD
              560 570 580 590 600
LLFKTNRKVT VKQLKEDYFK KIECFDSVEI SGVEDRFNAS LGTYHDLLKI
              610 620 630 640 650
IKDKDFLDNE ENEDILEDIV LTLTLFEDRE MIEERLKTYA HLFDDKVMKQ
              660 670 680 690 700
LKRRRYTGWG RLSRKLINGI RDKQSGKTIL DFLKSDGFAN RNFMQLIHDD
              710 720 730 740 750
SLTFKEDIQK AQVSGQGDSL HEHIANLAGS PAIKKGILQT VKVVDELVKV
              760 770 780 790 800
MGRHKPENIV IEMARENQTT QKGQKNSRER MKRIEEGIKE LGSQILKEHP
              810 820 830 840 850
VENTQLQNEK LYLYYLQNGR DMYVDQELDI NRLSDYDVDA IVPQSFLKDD
              860 870 880 890 900
SIDNKVLTRS DKNRGKSDNV PSEEVVKKMK NYWRQLLNAK LITQRKEDNL
              910 920 930 940 950
TKAERGGLSE LDKAGFIKRQ LVETRQITKH VAQILDSRMN TKYDENDKLI
              960 970 980 990 1000
REVKVITLKS KLVSDFRKDF QFYKVREINN YHHAHDAYLN AVVGTALIKK
            1010 1020 1030 1040 1050
YPKLESEFVY GDYKVYDVRK MIAKSEQEIG KATAKYFFYS NIMNFFKTEI
            1060 1070 1080 1090 1100
TLANGEIRKR PLIETNGETG EIVWDKGRDF ATVRKVLSMP QVNIVKKTEV
            1110 1120 1130 1140 1150
QTGGFSKESI LPKRNSDKLI ARKKDWDPKK YGGFDSPTVA YSVLVVAKVE
            1160 1170 1180 1190 1200
KGKSKKLKSV KELLGITIME RSSFEKNPID FLEAKGYKEV KKDLIIKLPK
            1210 1220 1230 1240 1250
YSLFELENGR KRMLASAGEL QKGNELALPS KYVNFLYLAS HYEKLKGSPE
            1260 1270 1280 1290 1300
DNEQKQLFVE QHKHYLDEII EQISEFSKRV ILADANLDKV LSAYNKHRDK
            1310 1320 1330 1340 1350
PIREQAENII HLFTLTNLGA PAAFKYFDTT IDRKRYTSTK EVLDATLIHQ
            1360
SITGLYETRI DLSQLGGD
```

In some embodiments, one or more, or all, of the DNA-binding domain, the activator domain, and the NLS are obtained or derived from a non-viral organism.

In some embodiments, the DNA-binding domain, the NLS, and the activator domain are linked in this order from N- to C-terminus.

A nucleic acid encoding the synthetic TF of any one of claims 1-54 operatively linked to a promoter capable of expressing the synthetic TF in vitro or in vivo.

A vector comprising the nucleic acid of the present invention.

In some embodiments, the vector is capable of stably integrating into a chromosome of a host cell or stably residing in a host cell.

In some embodiments, the vector is an expression vector.

A host cell comprising the vector of the present invention, wherein the host cell is capable of expressing the synthetic TF.

A system comprising a nucleic acid of the present invention and a second nucleic acid, or the nucleic acid, encodes a gene of interest (GOI) operatively linked to a promoter and one or more activator binding domains, or combination thereof, wherein the synthetic TF binds at least one of the one or more activator binding domain such that the synthetic TF modulates the expression of the GOI.

A genetically modified eukaryotic cell or organism, such as a plant cell or plant, comprising: (a) one or more nucleic acids each encoding one or more transcription activators operatively linked to a promoter; and (b) one or more nucleic acids each encoding one or more independent genes of interest (GOI) each operatively linked to a promoter that is activated by the one or more transcription activators; wherein at least one transcription activator is a synthetic transcription factor (TF) of the present invention.

In some embodiments, the promoter is a tissue-specific or inducible promoter.

In some embodiments, the transcription activator is the synthetic TF.

In some embodiments, any domain of the synthetic TF is heterologous to the eukaryotic cell or organism, such as a plant cell or plant, one or more of the GOI, any other transcription activator, and/or any of the promoters.

In some embodiments, the transcription activator is heterologous to the eukaryotic cell or organism, such as a plant cell or plant, one or more of the GOI, any other or transcription activator, and/or any of the promoters.

In some embodiments, the genetically modified plant cell or plant comprises: (a) a first nucleic acid encoding a transcription activator operatively linked to a first tissue-specific or inducible promoter; and (b) one or more nucleic acids each encoding one or more independent genes of interest (GOI) each operatively linked to a promoter that is activated by the transcription activators.

In some embodiments, each GOI is operatively linked to a promoter that is activated by the transcription activator.

In some embodiments, the promoter comprises one or more DNA-binding sites specific for the transcription activator. In some embodiments, the promoter comprises 1, 2, 3, 4, 5, 6, 7, 8, 9, or 10 DNA-binding sites specific for the transcription activator).

In some embodiments, the eukaryotic cell or organism is a plant cell or plant. In some embodiments, the eukaryotic cell or organism is a yeast. In some embodiments, the yeast is Saccharomyces species, such as a Saccharomyces cerevisiae.

### REFERENCES CITED

1. A. L. Sanborn, et al., Simple biochemical features underlie transcriptional activation domain diversity and dynamic, fuzzy binding to Mediator.eLife10 (2021).
2. Eukaryotic Transcription Factors—5th Edition (Feb. 2, 2022).
3. J. Tycko, et al., High-Throughput Discovery and Characterization of Human Transcriptional Activators. Cell 183, 2020-2035.e16 (2020).
4. G. Stampfel, et al., Transcriptional regulators form diverse groups with context-dependent regulatory functions. Nature 528, 147-151 (2015).
5. C. D. Arnold, et al., A high-throughput method to identify trans-activation domains within transcription factor sequences. EMBO J. 37 (2018).
6. A. Erijman, et al., A High-Throughput Screen for Transcription Activation Domains Reveals Their Sequence Features and Permits Prediction by Deep Learning. Mol. Cell 78, 890-902.e6 (2020).
7. M. V. Staller, et al., Directed mutational scanning reveals a balance between acidic and hydrophobic residues in strong human activation domains. Cell Syst. 13, 334-345.e5 (2022).
8. N. DelRosso, et al., Large-scale mapping and mutagenesis of human transcriptional activator domains. Nature 616, 365-372 (2023).
9. C. N. Ravarani, et al., High-throughput discovery of functional disordered regions: investigation of transactivation domains. Mol. Syst. Biol. 14, e8190 (2018).
10. Unable to find information for 13937619.
11. S. Jarriault, et al., Signalling downstream of activated mammalian Notch. Nature 377, 355-358 (1995).
12. J. Behrens, et al., Functional interaction of beta-catenin with the transcription factor LEF-1. Nature 382, 638-642 (1996).
13. J. C. Coates, L. Laplaze, J. Haseloff, Armadillo-related proteins promote lateral root development in Arabidopsis. Proc Natl Acad Sci USA 103, 1621-1626 (2006).
14. M. van de Wetering, et al., Armadillo coactivates transcription driven by the product of the Drosophila segment polarity gene dTCF. Cell 88, 789-799 (1997).
15. S. R. Kotha, M. V. Staller, Clusters of acidic and hydrophobic residues can predict acidic transcriptional activation domains from protein sequence. Genetics (2023).
16. M. V. Staller, et al., A High-Throughput Mutational Scan of an Intrinsically Disordered Acidic Transcriptional Activation Domain. Cell Syst. 6, 444-455.e6 (2018).
17. D. J. Glenn, R. A. Maurer, MRG1 binds to the LIM domain of Lhx2 and may function as a coactivator to stimulate glycoprotein hormone alpha-subunit gene expression. J. Biol. Chem.274, 36159-36167 (1999).
18. V. V. Ogryzko, R. L. Schiltz, V. Russanova, B. H. Howard, Y. Nakatani, The transcriptional coactivators p300 and CBP are histone acetyltransferases. Cell 87, 953-959 (1996).
19. S. Malik, M. Guermah, R. G. Roeder, A dynamic model for PC4 coactivator function in RNA polymerase II transcription. Proc Natl Acad Sci USA 95, 2192-2197 (1998).
20. Z. Liu, L. C. Myers, Fungal mediator tail subunits contain classical transcriptional activation domains. Mol. Cell. Biol. 35, 1363-1375 (2015).
21. N. F. C. Hummel, et al., The trans-regulatory landscape of gene networks in plants. Cell Syst. 14, 501-511.e4 (2023).
22. P. T. Monteiro, et al., YEASTRACT+: a portal for cross-species comparative genomics of transcription regulation in yeasts. Nucleic Acids Res. 48, D642-D649 (2020).
23. F. Tian, D.-C. Yang, Y.-Q. Meng, J. Jin, G. Gao, PlantRegMap: charting functional regulatory maps in plants. Nucleic Acids Res. 48, D1104-D1113 (2020).
24. K. Natter, et al., The spatial organization of lipid synthesis in the yeast Saccharomyces cerevisiae derived from large scale green fluorescent protein tagging and high resolution microscopy. Mol. Cell. Proteomics 4, 662-672 (2005).
25. W.-K. Huh, et al., Global analysis of protein localization in budding yeast. Nature 425, 686-691 (2003).
26. C. Hooper, et al., Subcellular Localisation database for Arabidopsis proteins version 5. The University of Western Australia (2022).
27. R. S. McIsaac, P. A. Gibney, S. S. Chandran, K. R. Benjamin, D. Botstein, Synthetic biology tools for programming gene expression without nutritional perturbations in Saccharomyces cerevisiae. Nucleic Acids Res. 42, e48 (2014).
28. B. K. Broyles, et al., Activation of gene expression by detergent-like protein domains. iScience 24, 103017 (2021).
29. A. M. Erkine, “nonlinear” biochemistry of nucleosome detergents. Trends Biochem. Sci.43, 951-959 (2018).
30. R. J. Emenecker, D. Griffith, A. S. Holehouse, Metapredict V2: An update to metapredict, a fast, accurate, and easy-to-use predictor of consensus disorder and structure. BioRxiv (2022).
31. A. J. Keung, C. J. Bashor, S. Kiriakov, J. J. Collins, A. S. Khalil, Using targeted chromatin regulators to engineer combinatorial and spatial transcriptional regulation. Cell 158, 110-120(2014).
32. I. Cherel, P. Thuriaux, The IFH1 gene product interacts with a fork head protein in Saccharomyces cerevisiae.Yeast 11, 261-270 (1995).
33. L. C. Myers, et al., The Med proteins of yeast and their function through the RNA polymerase II carboxy-terminal domain. Genes Dev. 12, 45-54 (1998).
34. D. E. Stemer, et al., Functional organization of the yeast SAGA complex: distinct components involved in structural integrity, nucleosome acetylation, and TATA-binding protein interaction. Mol. Cell. Biol. 19, 86-98 (1999).
35. H.-M. Bourbon, et al., A unified nomenclature for protein subunits of mediator complexes linking transcriptional regulators to RNA polymerase II. Mol. Cell 14, 553-557 (2004).
36. S. Tollis, et al., The microprotein Nrs1 rewires the GUS transcriptional machinery during nitrogen limitation in budding yeast. PLoS Biol. 20, e3001548 (2022).
37. M. G. Pray-Grant, J. A. Daniel, D. Schieltz, J. R. Yates, P. A. Grant, Chdl chromodomain links histone H3 methylation with SAGA- and SLIK-dependent acetylation. Nature 433, 434-438 (2005).
38. B. R. Cairns, et al., RSC, an essential, abundant chromatin-remodeling complex. Cell 87, 1249-1260(1996).
39. W. Czaja, P. Mao, M. J. Smerdon, Chromatin remodelling complex RSC promotes base excision repair in chromatin of Saccharomyces cerevisiae. DNA Repair (Amst)16, 35-43 (2014).
40. V. D., D. P., S. D., A role for Sds3p, a component of the Rpd3p/Sin3p deacetylase complex, in maintaining cellular integrity in Saccharomyces cerevisiae. Mol. Genet. Genomics 265, 560-568 (2001).
41. M. Papamichos-Chronakis, T. Petrakis, E. Ktistaki, I. Topalidou, D. Tzamarias, Cti6, a PHD domain protein, bridges the Cyc8-Tup1 corepressor and the SAGA coactivator to overcome repression at GAL1. Mol. Cell 9, 1297-1305 (2002).
42. X. Shen, G. Mizuguchi, A. Hamiche, C. Wu, A chromatin remodelling complex involved in transcription and DNA processing. Nature 406, 541-544 (2000).
43. S. Lanker, et al., Interactions of the eIF-4F subunits in the yeast Saccharomyces cerevisiae. J. Biol. Chem. 267, 21167-21171 (1992).
44. O. Matangkasombut, R. M. Buratowski, N. W. Swilling, S. Buratowski, Bromodomain factor 1 corresponds to a missing piece of yeast TFIID. Genes Dev. 14, 951-962 (2000).
45. L. Tora, A unified nomenclature for TATA box binding protein (TBP)-associated factors (TAFs) involved in RNA polymerase II transcription. Genes Dev. 16, 673-675 (2002).
46. N. L. Henry, et al., TFIIF-TAF-RNA polymerase II connection. Genes Dev. 8, 2868-2878 (1994).
47. Q. Xie, et al., LNK1 and LNK2 are transcriptional coactivators in the Arabidopsis circadian oscillator. Plant Cell 26, 2843-2857 (2014).
48. S. Kidokoro, et al., Clock-regulated coactivators selectively control gene expression in response to different temperature stress conditions in Arabidopsis. Proc Natl Acad Sci USA 120, e2216183120 (2023).
49. C. S. Gillmor, et al., The MED12-MED13 module of Mediator regulates the timing of embryo patterning in Arabidopsis. Development 137, 113-122 (2010).
50. C.-J. Wu, et al., Three functionally redundant plant-specific paralogs are core subunits of the SAGA histone acetyltransferase complex in Arabidopsis. Mol. Plant 14, 1071-1087 (2021).
51. K. Lee, P. J. Seo, The HAF2 protein shapes histone acetylation levels of PRR5 and LUX loci in Arabidopsis. Planta 248, 513-518 (2018).
52. Z. Lai, et al., Arabidopsis sigma factor binding proteins are activators of the WRKY33 transcription factor in plant defense. Plant Cell 23, 3824-3841 (2011).
53. A. M. Erkine, D. S. Gross, Dynamic chromatin alterations triggered by natural and synthetic activation domains. J. Biol. Chem. 278, 7755-7764 (2003).
54. M. Abedi, et al., Transcriptional transactivation by selected short random peptides attached to lexA-GFP fusion proteins. BMC Mol. Biol. 2, 10 (2001).
55. J. Ma, M. Ptashne, A new class of yeast transcriptional activators. Cell 51, 113-119 (1987).
56. L. Moffat, D. T. Jones, Increasing the Accuracy of Single Sequence Prediction Methods Using a Deep Semi-Supervised Learning Framework. Bioinformatics 37, 3744-3751 (2021).
57. G. Erdös, M. Pajkos, Z. Dosztinyi, IUPred 3: prediction of protein disorder enhanced with unambiguous experimental annotation and visualization of evolutionary conservation. Nucleic Acids Res. 49, W297-W303 (2021).
58. R. Daniel Gietz, R. A. Woods, “Transformation of yeast by lithium acetate/single-stranded carrier DNA/polyethylene glycol method” in Guide to Yeast Genetics and Molecular and Cell Biology—Part B, Methods in Enzymology, (Elsevier, 2002), pp. 87-96.
59. D. C. Amberg, J. N. Strathern, Methods in Yeast Genetics: A Cold Spring Harbor Laboratory Course Manual (CSHL Press)(2005).
60. M. S. Belcher, et al., Design of orthogonal regulatory systems for modulating gene expression in plants. Nat. Chem. Biol. 16, 857-865 (2020).
61. S. Piskacek, et al., Nine-amino-acid transactivation domain: establishment and prediction utilities. Genomics 89, 756-768 (2007).
62. R. J. Emenecker, D. Griffith, A. S. Holehouse, Metapredict: a fast, accurate, and easy-to-use predictor of consensus disorder and structure. Biophys. J. 120, 4312-4319 (2021).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

Systematic Identification of Transcriptional Activator Domains from Non-Transcription Factor Proteins in Plants and Yeast

Transcription factors activate gene expression via trans-regulatory activation domains. Although activation domains have been cataloged at whole genome scales in model organisms (e.g. human, yeast, fly), their occurrence in non-transcription factor proteins have not been systematically explored. Transcriptional coactivators, chromatin regulators and some cytosolic proteins contain functional activation domains leaving a blind spot on the occurrence of activation domains in these proteins. Therefore, the activation domain predictor PADDLE is utilized to mine the entire proteomes of two model eukaryotes, Arabidopsis thaliana and Saccharomyces cerevisiae (1). 18,000 fragments covering predicted activation domains from >800 non-TF genes in both species, and experimentally validated that the majority (78%) of proteins contained fragments capable of activating transcription in yeast are characterized. The results show that divergent activity of activation domains of similar sequence composition can be largely explained by positional distribution of key amino acid residues. Hundreds of nuclear proteins with activation domains as putative coactivators in both systems are annotated, establishing the first coactivator map for plants. Furthermore, the library contained >250 non-nuclear proteins containing activation domains across both eukaryotic lineages, suggesting that there are unknown biological roles that these peptides may serve beyond facilitation of transcription. Finally, ‘universal’ eukaryotic activation domains that activate transcription in both systems with comparable or stronger performance to state-of-the-art activation domains are establish. Overall, activation domains in non-transcription factor genes throughout the proteomes of two distantly related eukaryotes are annotated and the results will help unravel the function of these diverse peptides.

Here, the capability of ADs derived from non-TF proteins to promote transcription when utilized in synthetic TFs is explored. A library of 18,000 synthetic TFs based on predicted ADs from non-TF genes with ADs derived from S. cerevisiae and A. thaliana is generated. It is shown that 77% of genes in the library contain ADs capable of promoting transcription in yeast with some candidates showing stronger activation than benchmark activators. ADs in nuclear genes associated with known coactivator and chromatin regulator activity, and in genes previously unassociated with the activation of transcription are found. Notably, ADs were not limited to nuclear genes and many ADs were found in a wide range of protein families localized to non-nuclear organelles. Furthermore, one finds positional distribution of key amino acids that make large contributions to AD activity and show how strong ADs from the library activate transcription in plants but correlate weakly with their activity in yeast. The large interspecies dataset provides the foundation to better characterize the sequence space in non-TFs and identify new proteins potentially involved in transcriptional activation across distantly related eukaryotes.

### Results:

Characterization of a Library of Non-TF ADs Mined from Yeast and Plant Proteomes

It was aimed to systematically discover previously uncharacterized ADs derived from non-TF proteins in two model eukaryotic systems, A. thaliana and S. cerevisiae. In previous work, it has been shown that AD predictors derived from fungal data can accurately predict ADs in plant TFs and that plant ADs function in yeast (21). Here, this result is leveraged to predict activators in plant and yeast proteins, followed by high-throughput experimental validation in yeast.

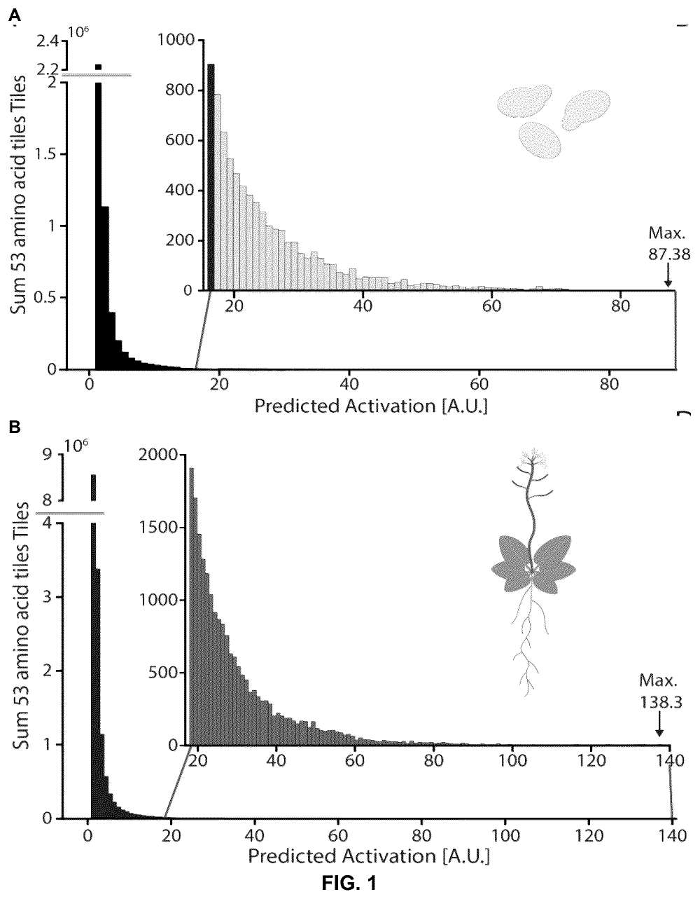

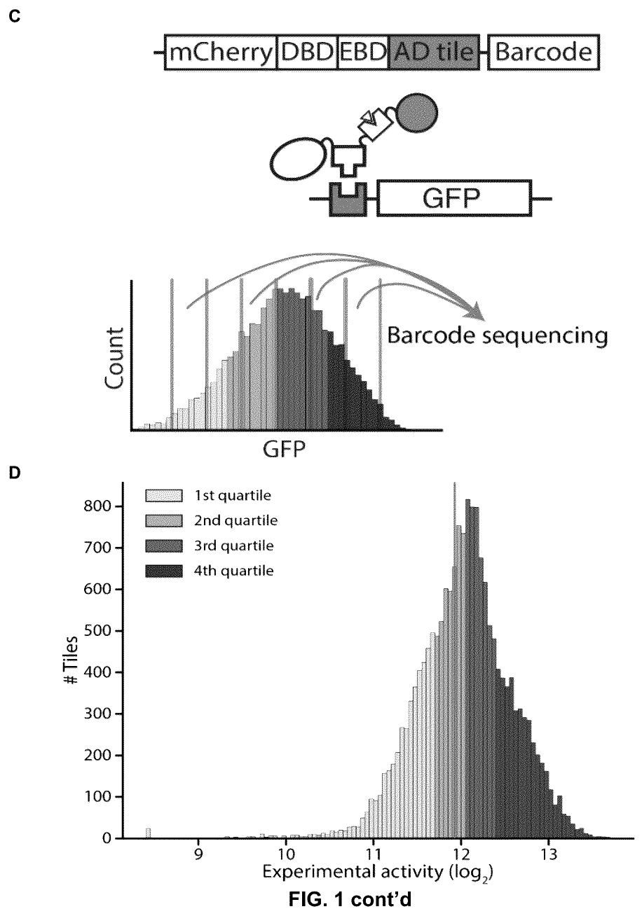

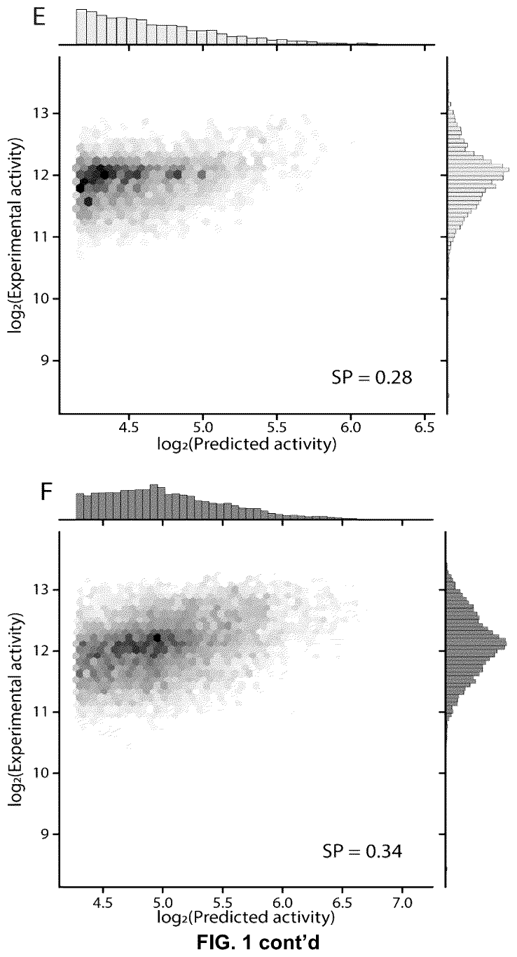

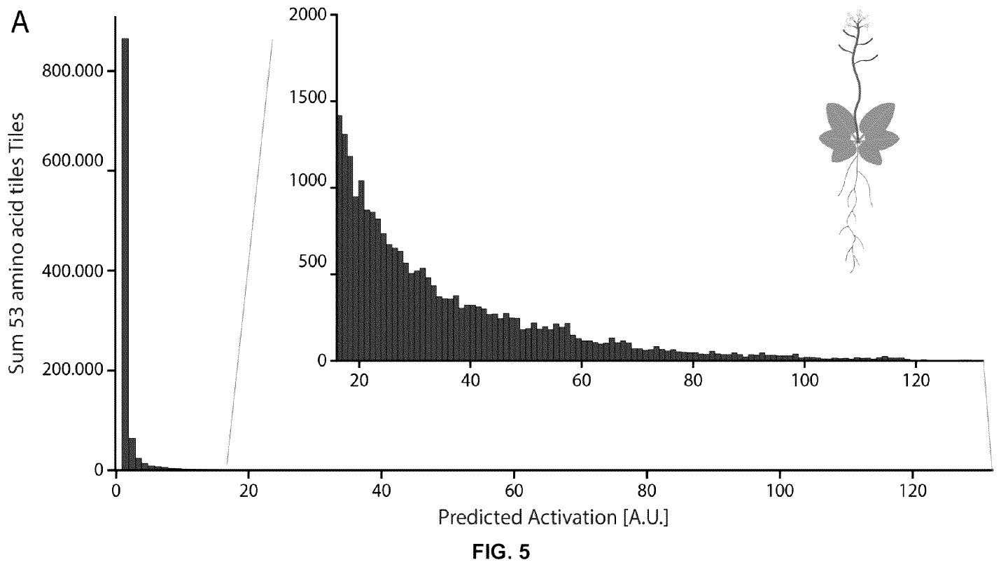

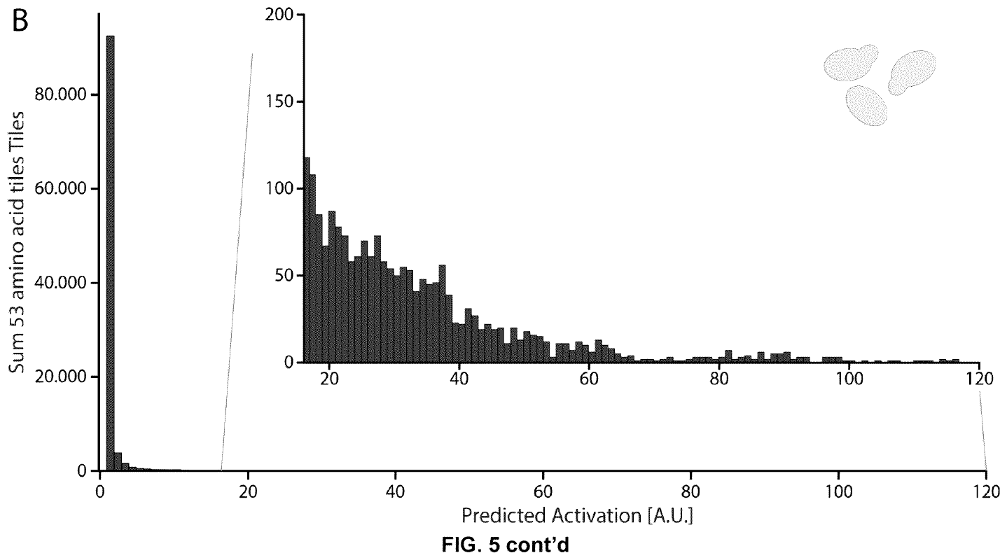

To extract potential ADs from both proteomes, PADDLE, a neural network model capable of predicting acidic ADs in 53 amino acid long peptides (1) is utilized. Each proteome is computationally chopped in 53 AA tiles spaced every one residue, yielding at least 9,211,910 tiles in A. thaliana and at least 2,646,422 tiles in S. cerevisiae derived from at least 27,082 and 6,455 proteins, respectively (FIG. 1, panels A and B, Supplementary Data 1). PADDLE is used to predict the potential of all tiles to activate transcription. Then TF databases for both species are used—PlantTFDB v5.0 and Yeastract+—to remove all tiles derived from TF sequences (22, 23). We found that tiles from non-TF genes had a similar dynamic range of predicted activity as tiles from TF genes (FIG. 5, panels A and B) and in Arabidopsis the strongest predicted tile occurred in a non-TF gene (AT5G07570.1). Then 12,000 tiles from A. thaliana and 6,000 tiles from S. cerevisiae with the highest predicted activation score are selected, yielding an 18,000-tile library derived from 447 Arabidopsis and 402 yeast parent proteins, respectively. A parent gene is defined as the underlying protein sequence that all tiles are generated from, as multiple tiles can come from a single parent gene. It was chosen to include overlapping tiles to increase accuracy and resolution. To gauge the subcellular localization of parent proteins of the library, SUBA5 for Arabidopsis and YeastGFP/YPL+ is utilized to annotate localization (FIG. 1, panel C) (24-26). There was a total of 209 parent proteins localized to the nucleus in Arabidopsis and 92 in yeast and non-nuclear genes were localized throughout all organelles in both species (Supplementary Table 1, 2). This diversity of localization suggests that peptides predicted to be ADs occur throughout various organelles across both proteomes.

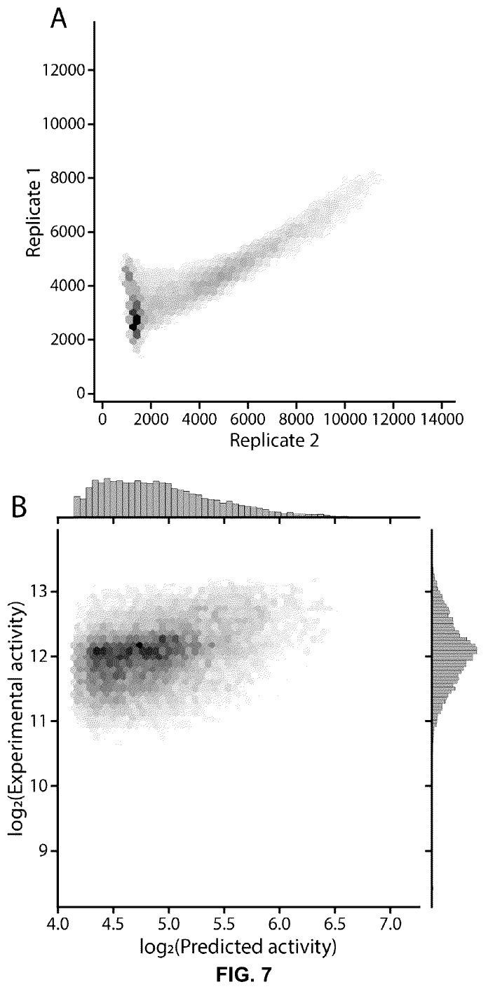

To experimentally characterize and validate the library, a previously established expression system utilizing synthetic TFs in yeast is used (16) (FIG. 1, panel C). In this expression system each tile is fused to a synthetic TF, consisting of 1) mCherry for normalization of TF concentration to generated reporter signal as a N-terminal fusion, 2) the orthogonal murine Zif268 DBD for TF localization, 3) a human estrogen response domain to make the system inducible with ß-estradiol, 4) the 53 amino acid long AD candidate, and 5) a unique barcode in the 3′ UTR marking candidate identity in the library. The associated reporter consists of six copies of the Zif268 binding sites upstream of a modified GAL1 promoter driving GFP (27). Both the reporter and the synthetic TF were integrated into the genome of S. cerevisiae to reduce expression variability. Fluorescence-activated cell sorting (FACS) is used to sort the library (see Methods) and experimentally validated the activity of at least 17,553 tiles (97.5% of total library) from at least 846 parent genes with high reproducibility between replicates (FIG. 7, panel A, Supplementary Table 3, 4, Pearson's r=0.82). The approach that includes multiple DNA barcodes per tested tile further increases accuracy of measurements as previously shown (16).

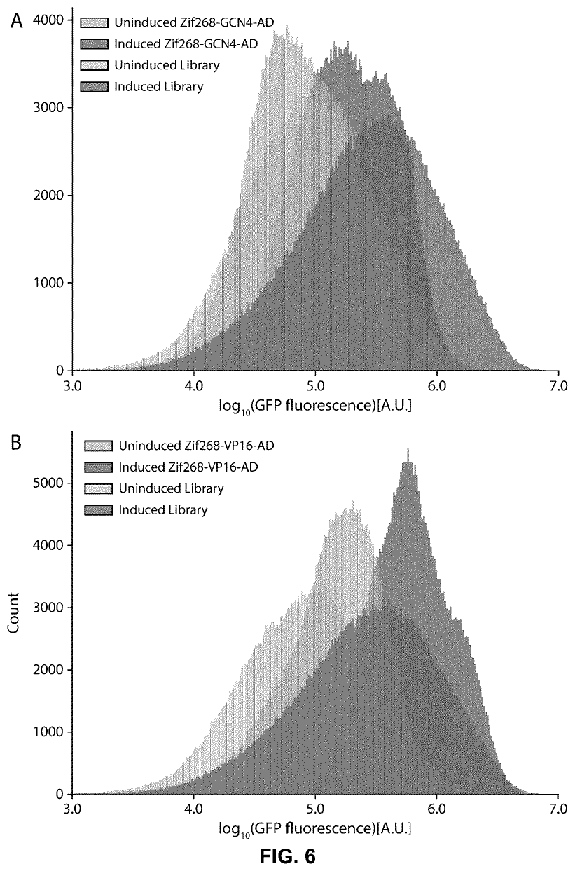

The experimental activity of the library allowed one to evaluate the accuracy of PADDLE predictions. In the PADDLE training dataset, ˜6% of TF derived tiles showed activity and the model achieved reliable qualitative and quantitative prediction of ADs (Pearson's r=0.80). In the library, tile activity ranged over three orders of magnitude with 50% of the library showing significant activity above no-TF control levels (FIG. 1, panel D). This was the largest fraction of active tiles that was observed using this system (Staller et al. 2018; Staller et al. 2022). Parts of the library activated transcription equally or stronger than ZIF268-GCN4-AD and -VP16-AD controls (FIG. 6, panels A and B). It was found that 76.1% of parent genes (644 out of 846) of tiles in the library contain at least one tile with activator activity, demonstrating that tiles that can function as ADs are widespread throughout non-TF genes. This result shows how PADDLE can in most cases correctly localize ADs in proteins, but that its architecture, which predicts AD likelihood and then extrapolates activity predictions, is not rigorous enough to predict both qualitative and quantitative aspects of ADs.

### Single Amino Acid Tiling Unravels Novel Key Residues in AD Activity

Distribution of key amino acid groups dictates AD activity. While the large fraction of active tiles supports the ability of PADDLE to localize ADs in protein sequences, the quantitative predictions of AD strength did not correlate as strongly with the experimental results (FIG. 7, panel B, Spearman's r=0.35). Notably, the PADDLE algorithm predicted activity with higher accuracy in A. thaliana than in S. cerevisiae AD populations with moderate Spearman correlation coefficient of 0.34 and 0.28, respectively (FIG. 1, panels E and F), and in 13% of parent genes did the strongest tile correlate with the highest PADDLE prediction. Hence, PADDLE correctly identifies the general location of ADs but struggles to accurately predict the quantitative strength of the respective AD. The results suggest that there are likely positional effects of amino acid residues dictating AD activity that PADDLE cannot resolve.

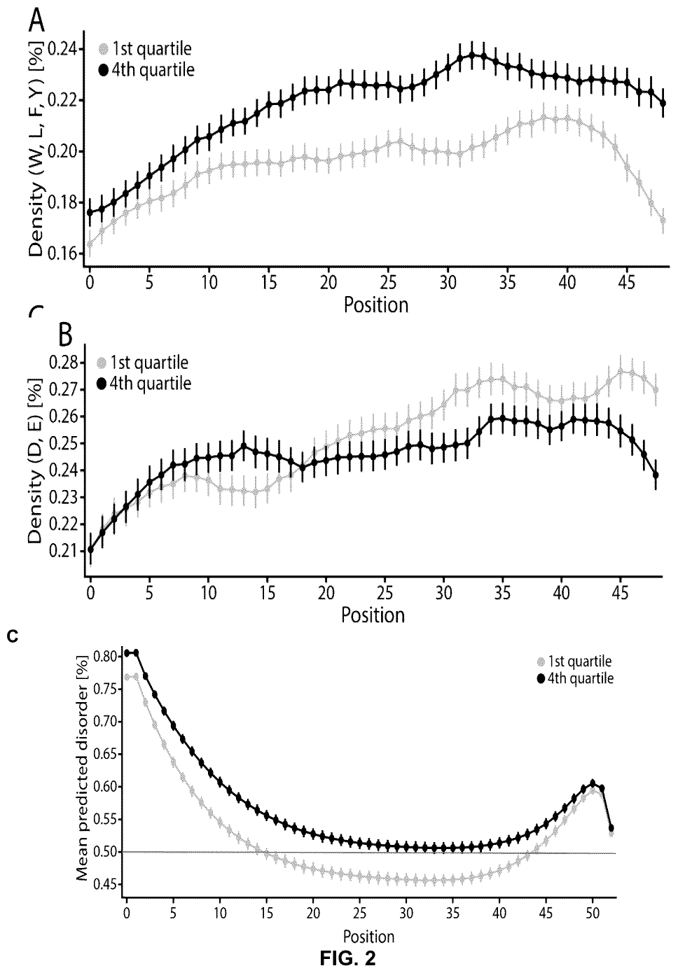

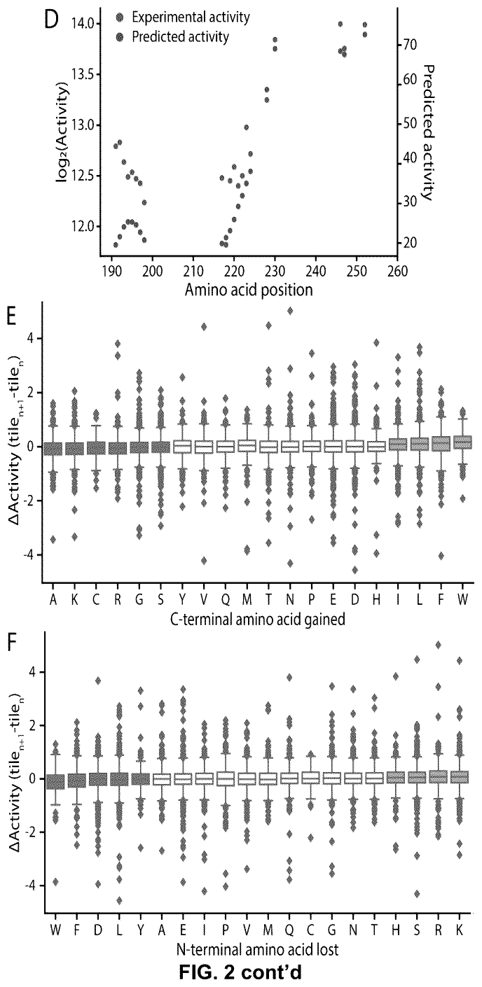

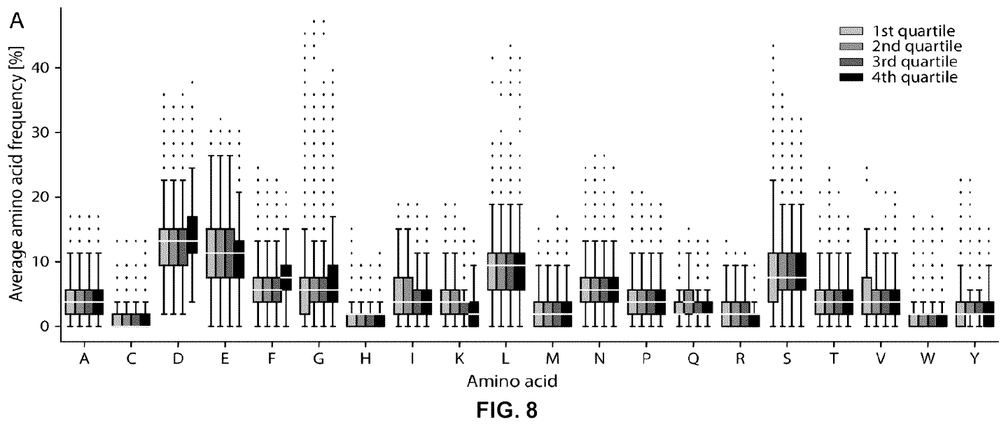

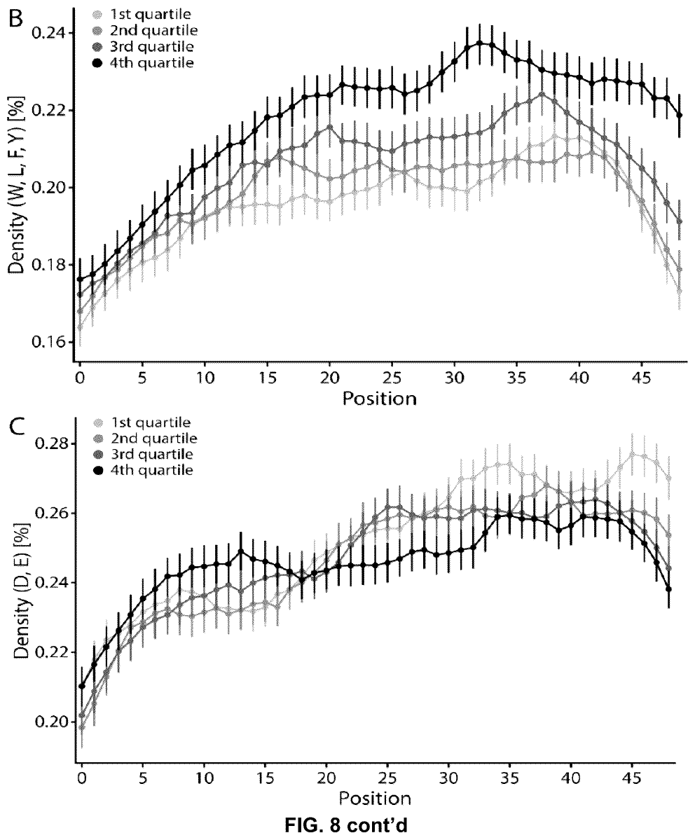

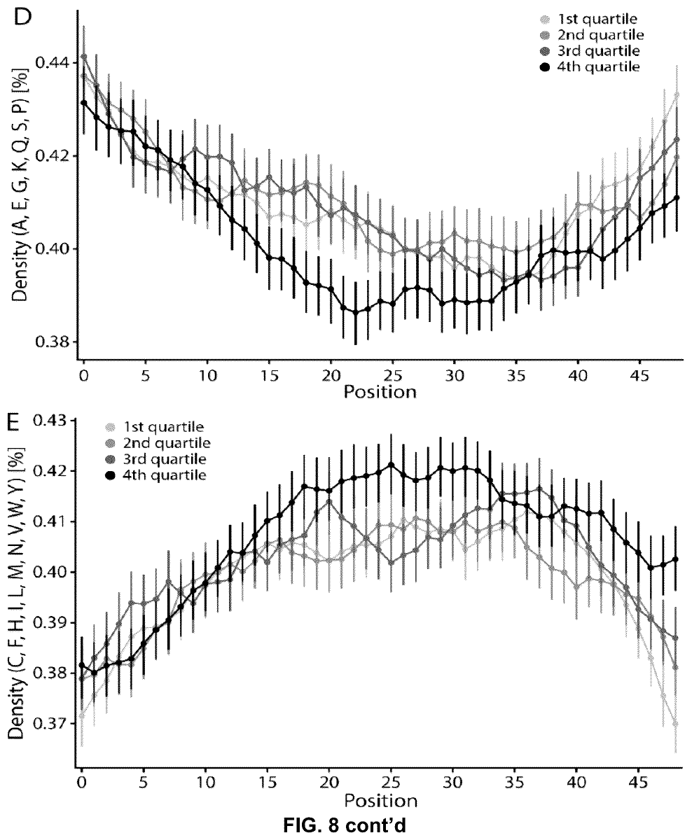

To further investigate the discrepancies between PADDLE predictions and observed activities, it is examined how amino acid composition and positional context may play a role in defining AD activity. Previous studies have lacked this resolution and still have been able demonstrated that specific amino acid residues (i.e., D, F, W, L) and specific dipeptides are linked to stronger activator activity (1, 6, 28, 29), but they have not been able to discern how the positional grammar of functional amino acids along the AD influences activity. To compare tile populations, the entire library is split into four equal quartiles reaching from weakest to strongest activity. Notably, when compared the average amino acid composition of unilength tiles in all four quartiles, it was almost identical (FIG. 8, panel A). Thus, it was hypothesized that amino acid composition alone cannot explain AD activity and that the positional distribution of functional amino acids is key. Hence, to gauge the positional information encoded in each quartile, the local density of all residues along each tile of every quartile is measured, where density is the frequency of the respective amino acid in a 5 amino acid window. Then amino acid groups linked to AD activity and computed density at each position of the 53 amino acids of every tile of each respective quartile were grouped. It was found that the density of hydrophobic residues, which are linked to AD activity, to be overall higher in the highest activity quartile, and density was higher in the C-terminus when compared to tiles in other quartiles (FIG. 2, panel A; FIG. 8, panel B). All quartiles had low density of hydrophobic residues in the N-terminus suggesting that the localization of hydrophobic residues close to the C-terminus is relevant for activity. In the 4th quartile, acidic residues showed a weaker density in the C-terminus and a more even distribution throughout the entire tile, whereas the weaker quartiles had a stronger enrichment of acidic residues in the C-terminus and depletion in the N-terminus (FIG. 2, panel B; FIG. 8, panel C). These results support the hypothesis that the occurrence of key amino acids—in this case hydrophobic and acidic residues—alone does not correlate with activity but rather their distribution along the AD, supporting the previously proposed acidic exposure model (7).

The structure of tiles in the library was further studied. To gauge the disorder of tiles in the respective quartile, the disorder predictor Metapredict V2 was utilized (30). It was found that all quartiles displayed increased disorder in their N-terminus, suggesting that initial disorder in the tile is important for activity (FIG. 2, panel C). In all quartiles disorder dropped drastically in the C-terminus and the 4th quartile showed increased disorder throughout the entire tile staying above the disorder threshold >0.5 in comparison to all quartiles, except for the C-terminus (Fig. S6 of Supplemental Information). The results indicate that disorder in the N-terminus is essential for AD activity and induces overall disorder in ADs. Acidic residues throughout the tile prevent the accumulated order promoting residues from the intramolecular collapse of the disordered peptide into a structured state, another feature of the acidic exposure model. It is proposed that local density enrichment of functional amino acid groups dictates AD activity and peptide composition alone doesn't suffice to predict activity. Taken together, positional biases visible in tile populations that should be of similar activity based on PADDLE predictions highlights the weakening predictive power of AD activity in stronger ADs by PADDLE; suggesting that current predictive models are still missing necessary positional information of key residues in ADs that need to be incorporated into network training.

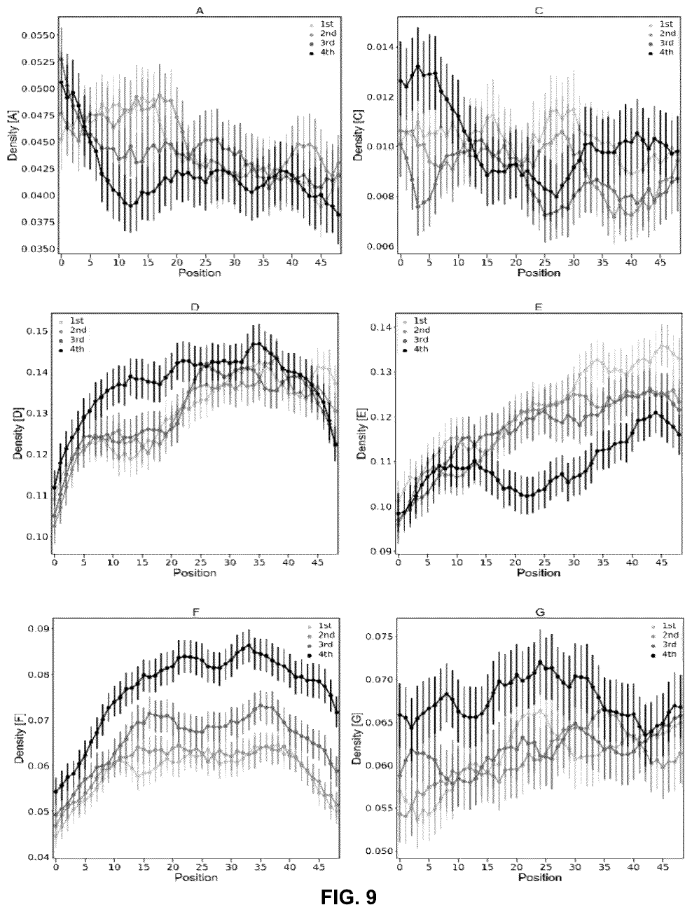

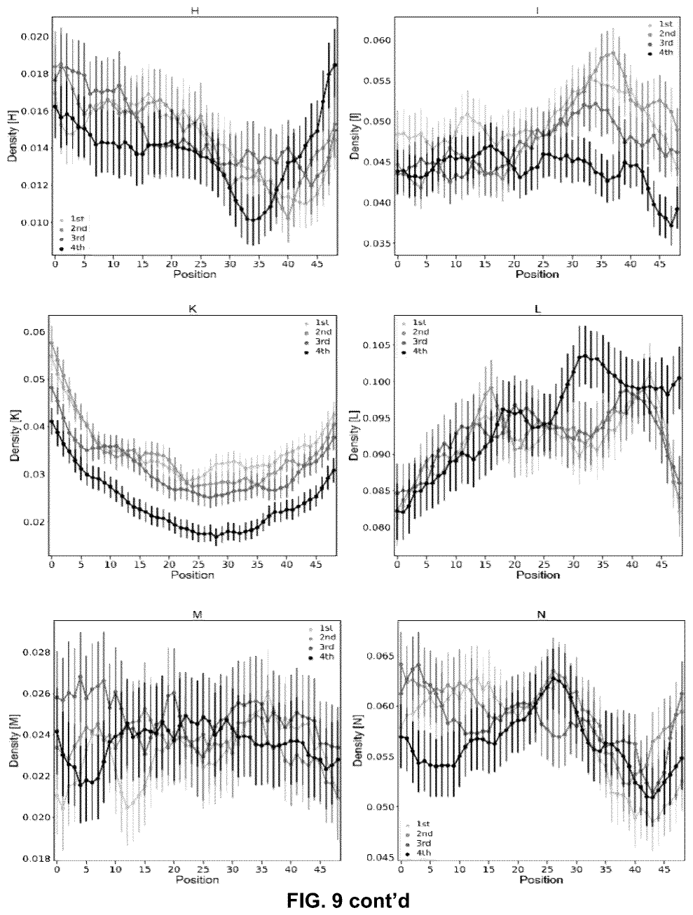

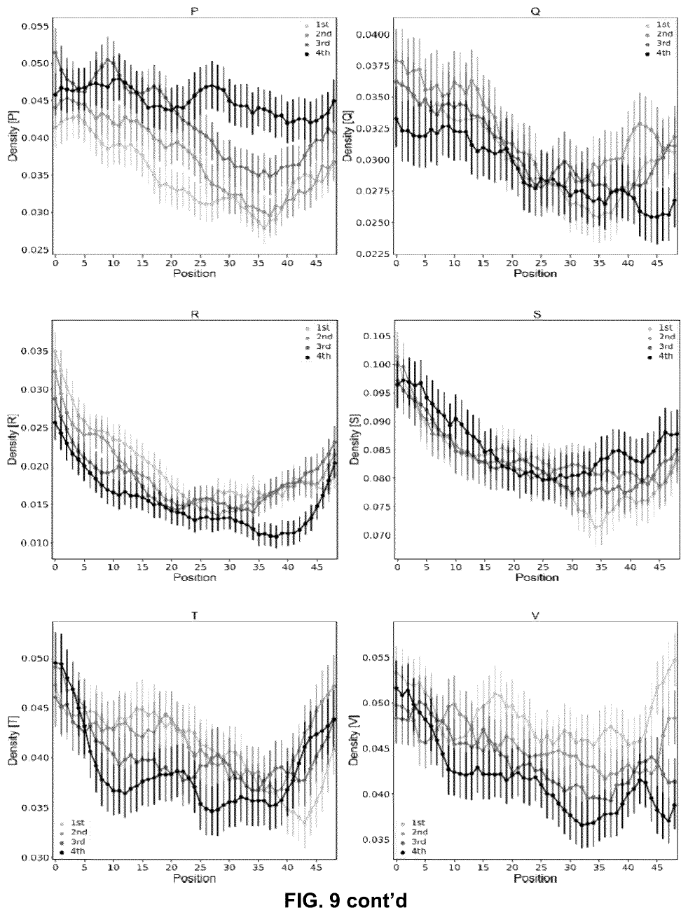

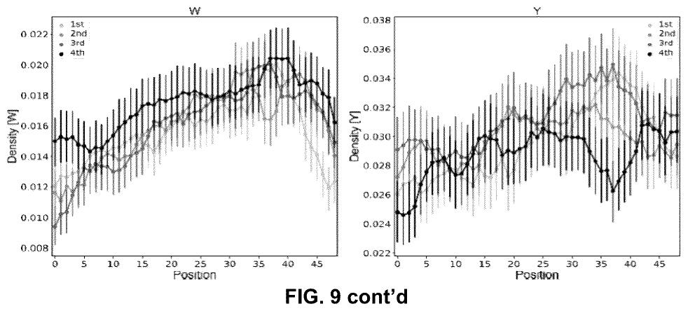

High-throughput studies usually scan protein sequences when performing tiling, only covering fragments of a protein in step sizes between 5-10 amino acids. It was decided to tile at single amino acid resolution to study how single amino acid changes both from losing and gaining one amino acid during tiling affects AD activity (FIG. 2, panel D). At the C-terminus, gaining the hydrophobic residues (W, F, L) enhanced activity as expected. Notably, isoleucine, which is not normally linked to enhancing activity, had a stronger positive effect on activity than acidic residues (FIG. 2, panel E). The enrichment of isoleucine was only observed in the C-terminal position tiles (FIG. 9), suggesting an unknown role of this residue in AD activity. At the N-terminus all effects were smaller than for the C-terminus, but losing hydrophobic residues or aspartate had a negative effect on activity, and losing positively charged residues arginine and lysine increased activity following known rules of activity (FIG. 2, panel F). Overall, for all observations there was a large spread between observed effects, showcasing how all changes to the amino acid composition and distribution are context dependent and general frequencies of residues can only be utilized as a guide post for gauging AD activity.

### A Genome-Wide Compendium of Coactivator Candidates in Plants.

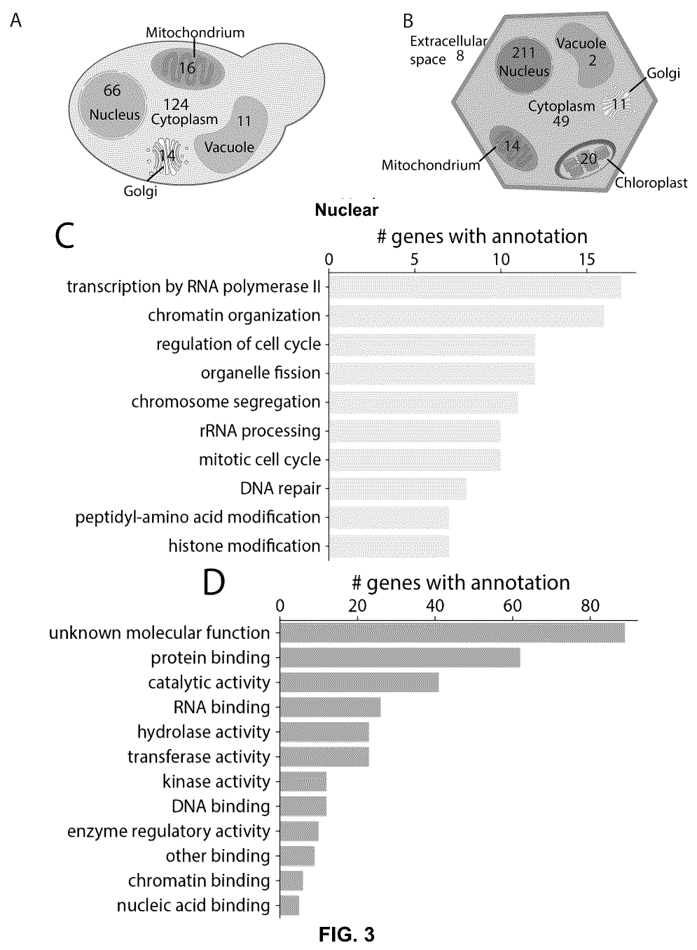

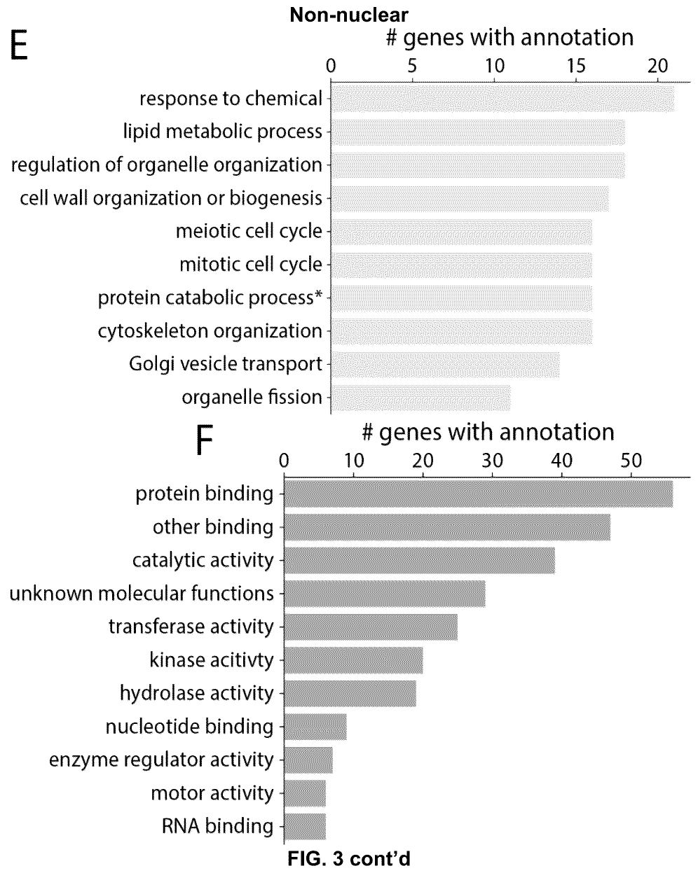

Coactivators provide an interface between TFs and RNA polymerase and are essential for the activation of gene expression. Although there has been significant attention on characterizing TFs at a genome-scale, only a limited number of coactivators have been characterized in plants, limiting one's ability to fully understand how they affect transcriptional regulation. Recent studies have shown that coactivators and chromatin regulators can directly modulate transcription when localized to DNA independent of TFs and that they contain ADs (4, 31). Hence, ADs can occur in any gene involved in transcription; thus, nuclear non-TF genes with ADs are potential coactivators. Based on subcellular localization data, the library contains ADs from 101 and 211 nuclear non-TF genes in yeast and Arabidopsis, respectively, allowing one to explore their potential coactivator function (FIG. 3, panels A and B).

Coactivators have been more thoroughly studied in yeast than in plants; hence, the occurrence of ADs in nuclear non-TF genes from yeast was benchmarked both to identify previously unannotated coactivator candidates as well as provide a more comprehensive list of known coactivators with ADs. To ensure only annotating genes with functional ADs, parent genes that yielded the 50% strongest ADs in the library were included. Gene ontology (GO) terms were used to gauge the function of candidate genes and found most GO terms to be linked to transcription like ‘transcription by RNA polymerase II’, ‘chromatin organization’, ‘regulation of cell cycle’ and ‘histone modification’ (FIG. 3, panel C). As expected, tiles derived from known coactivators in yeast, namely IFH1, MED2, ROX3 and NRS1 were characterized (32-36). It was also found that ADs in chromatin regulators, namely HFI1, CHD1, SFH1, STH1, SDS3, CTI6 and INO80 (34, 37-42). Tiles from other proteins involved in transcription included transcription initiation factor eIF4G1, TATA binding factor TAF1, TAF14 and BDF1 as well as general TF TFG1 (43-46). Notably, one found ADs in two genes of unknown protein family and function (YBL029W, YML108W) which may function as potential co-activators. Candidate genes were also associated with the GO terms ‘rRNA processing’ and ‘chromosome segregation’, raising the question what roles ADs might play in these proteins. Overall, previous observations of ADs in coactivator complexes and chromatin regulators were supported by the results, suggesting that the approach could be used to identify genes with putative regulatory function from gene families that are not commonly associated with transcription.

Coactivators in plants have been far less studied and mostly annotated based on homologs from other eukaryotes (mediator ref). Hence, the parent genes of tiles from Arabidopsis contained far fewer hits in known transcription associated genes. Of the 211 nuclear non-TF hits, only 4 had previously been validated to be coactivators, highlighting the opportunity to discover new plant coactivators. It was found that ADs in the 4 previously studied coactivators MED13 and LNK1/LNK2/LNK3 (47-49), the chromatin regulators HAF2 and SCS2A/B (50, 51), and four transcription elongation factors from family S-II. It was also found that ADs in seven genes that have been annotated but not experimentally validated as transcriptional coactivators. For example, 3 members of the VQ family of suspected transcriptional coregulators that interact with WRKY family TFs during abiotic stress response contain ADs in the library (52). Four CCT-motif-containing proteins that have been linked to transcriptional elongation in other eukaryotes were also represented. Notably, only a few GO terms were linked to transcription, with a total of 23 genes linked to potentially transcription associated GO terms like ‘chromatin binding’, ‘nucleic acid binding’, ‘DNA binding’. The most abundant GO term was ‘unknown molecular functions’ with 89 associated genes, highlighting the high probability of putative coactivators that likely have not yet been characterized (FIG. 3, panel D). Other nuclear genes with ADs were either not previously associated with transcription or have never been studied before, suggesting that there may be plant-specific coactivators that cannot be annotated purely based on sequence homology to other eukaryotes. The results supply an extensive list of putative coactivators in Arabidopsis which should accelerate their proper characterization and help identify potential key players involved in plant gene regulation.

### Non-Nuclear Proteins Throughout all Organelles Contain Strong Ads

Non-nuclear proteins can contain ADs and influence transcription via relocation to the nucleus as has been observed in the examples of Notch1 and beta-catenin (11, 12). The role of ADs that occur in proteins outside of the nucleus was explored. Focus on parent genes harboring tiles of the 50% strongest experimentally validated ADs, yielding 136 Arabidopsis and 149 yeast non-nuclear genes. It was found that 46 and 70 cytosolic in Arabidopsis and yeast, respectively. While these genes are candidates to be relocalized to the nucleus to facilitate transcription, there were 90 Arabidopsis and 79 yeast non-nuclear, non-cytosolic genes that are distributed throughout all organelles and membranes (FIG. 3, panels A and B). To gauge their role, the GO terms of all non-nuclear parent genes of both species were studied. In yeast, GO terms were unrelated to transcription and included both metabolic terms like ‘lipid metabolic process’, signaling terms like ‘response to chemical’. Many GO terms were linked to architecture of the cell like ‘meiotic/mitotic cell cycle’, ‘cytoskeleton organization’ and ‘organelle fission’ (FIG. 3, panel E). In Arabidopsis, the two most abundant molecular function terms were linked to “protein binding” and “general binding”, followed by “catalytic activity” and “unknown molecular function” (FIG. 3, panel F). This highlights the diverse functionalities of non-nuclear proteins with AD-like sections in both species. The bias towards GO terms linked to binding interactions, raises the question whether AD-like peptides can facilitate protein-protein interactions outside of the nucleus.

### A New Set of Universal Eukaryotic Activation Domains

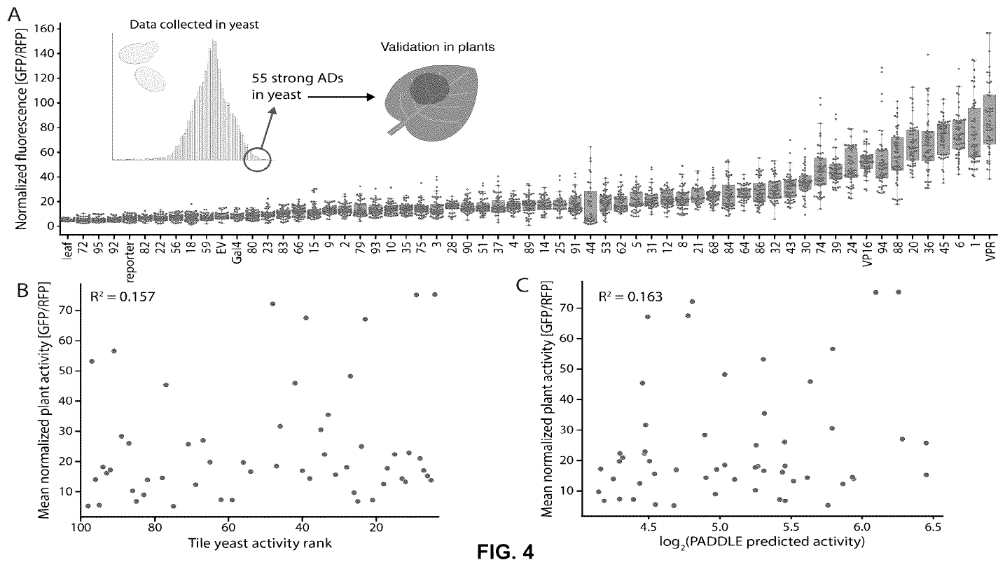

The library provides the unique opportunity to validate the transferability of yeast derived ADs into plants and establish potential universal activators that function in phylogenetically divergent eukaryotes. Therefore, 55 of the strongest ADs in the library—33 derived from Arabidopsis and 22 from yeast—in the plant N. benthamiana were agnostically characterized using an agroinfiltration mediated transient expression system that was previously established (21). In this system each tile is fused to the yeast GAL4-DBD and localized to a synthetic minimal promoter with 5 concatenated GAL4 binding sites to drive GFP (FIG. 4, panel A), modulating GFP expression. A constitutively expressed dsRed is used to normalize the signal. To control the potency of the AD tiles, the strong activator VP16 and VPR (a fusion of the three strong activators VP64, p65 and Rat) as GALA fusions were also tested. Of 55 ADs tested, it was found that 43 (78.2%) to significantly increase GFP expression over the reporter only control, 6 were stronger than VP16 and 2 of similar activity as VPR (FIG. 4, panel A, verified by Mann-Whitney-U test, p<0.05). Notably, it was found that tested ADs to range the entire dynamic range of possible activities, from no observed to very strong activity, highlighting the importance of cross-validation even if ADs showed strong activity in yeast. Overall, it was discovered that short, universal ADs from non-TF proteins that perform similarly to longer state of the art ADs like VPR that are readily available for further eukaryotic engineering efforts.

The agnostic approach to mapping ADs in non-TF genes allowed one to mine strong ADs from proteins that have not previously been associated with transcription and it was shown that these ADs function in plants. As an example, ADs in known plant coactivators, namely one CCT-motif containing protein from Arabidopsis (AT1G04500), coactivator LNK3 (AT3G12320) and SAGA complex subunit 2A (AT2G19390), which showed activity similar to the VP16 control were localized and validated. Furthermore, the strongest AD in plants was derived from an Arabidopsis uncharacterized 2Fe-2S ferredoxin-like superfamily protein (AT1G50780). The second strongest AD was derived from a hypothetical protein (AT2G29920). These results support the mapping of unstudied genes as putative coactivators as well as localizing ADs in known coactivators using this approach.

Eukaryotic TFs utilize conserved general transcription machinery (e.g., Mediator) to facilitate transcription, making new TF parts a potential resource to develop tools for the control of transcription across eukaryotes. For the plant experiments only the strongest tiles from the yeast experiments were chosen, expecting that, if they utilize general conserved transcription machinery, activities in plants should be similarly strong. Against expectation it was found that poor correlation between rank order of yeast AD rank and plant ranking (FIG. 4, panel B). Furthermore, PADDLE predictions correlated worse with observed AD activity in plants than in yeast (FIG. 4, panel C). The results suggest that, while PADDLE can localize ADs in both plant and yeast proteins with 78.2% accuracy, there are mechanistic features of plant transcription not fully captured by PADDLE that prevent accurate prediction of AD strength. It was concluded that it is necessary to generate independent plant AD datasets to train neural networks that can predict the strength of plant ADs with higher accuracy to enable the full potential of mining plant proteomes.

### DISCUSSION

High-throughput studies have largely focused on ADs found in TFs and protein classes known to be involved in transcription, which has partly biased the understanding of the biological role of such peptides. By mining proteomes for ADs from non-TF genes and demonstrating their activity in yeast and plants, it was revealed that ADs frequently occur across entire proteomes and outside the nucleus, going beyond the canonical description of ADs in TFs that mediate nuclear transcription. Studying nuclear non-TF ADs from the well-studied model yeast expands the understanding of which genes contain AD-like peptides and where they are localized. It was found that a direct correlation between nuclear genes containing ADs and their likelihood to function as coactivators in yeast. The dataset provided the motivation to extrapolate this observation to plants and an exhaustive list of 200 putative coactivators that may be involved in many facets of plant transcriptional regulation was annotated. Due to the throughput limitations of the experimental setup, focus was set on the strongest 18,000 tiles from both species, leaving a far larger sequence space of medium or weak ADs unstudied. Future work will focus on experimentally validating larger sets of predicted ADs in both species and help understand how frequent ADs occur throughout proteomes.

The recent establishment of large experimental datasets of activation domains in yeast has led to the development of multiple neural networks that attempt to localize and predict the activity of ADs from protein sequences (1, 6). In this study one of these models PADDLE was utilized to build and test the library. It was found that PADDLE can correctly localize ADs throughout entire proteomes; however, the capabilities of PADDLE to predict the quantitative activity of ADs fell short in comparison to the high correlation value that was reported in the study that implemented it. It was further shown that plant tiles that functioned as strong ADs in yeast, indeed largely functioned in plants but only few ADs showed regulatory activity similarly to their activity in yeast. This discrepancy indicates that while general eukaryotic mechanisms for the regulation of transcription between plants and yeast are conserved, there are intricacies in plants that models trained on yeast data cannot resolve. It further highlights that the flexible positional and compositional sequence requirements of ADs need to be explored further in their native context. The current state of the art models can be utilized to rapidly discover strong ADs for engineering in plants but their full potential has yet to be fulfilled and will allow a simple pipeline for mining entire plant proteomes for ADs. Furthermore, the non-TF centric approach yields a far larger sequence space of peptides providing a near unlimited number of ADs readily available to test for plant genome engineering efforts.

At their core, ADs facilitate protein-protein interactions with transcriptional machinery to facilitate transcription. Recent studies have shown that these interactions rely on multiple weak interactions between disordered ADs and folded machinery and up to 1% of random sequences have AD activity when localized to promoters (53-55). This knowledge combined with the observed abundance of peptides with AD-like properties throughout entire proteomes in two distantly related eukaryotes, leads one to the conclusion that ADs are likely a simple class of protein-protein interaction modules. These modules facilitate transcription inside the nucleus, while in a non-nuclear context they still mediate protein-protein interaction but with other functional outcomes. Especially in distinctly physically separated organelles like mitochondria and chloroplasts these interactions could lead to a different regulatory outcome independent of transcription. In summary, the versatile nature of ADs extends their role beyond nuclear transcription and blurs the distinction of ADs as a feature unique to TFs.

### Material and Methods

PADDLE Prediction of Every 53 Amino Acid Tile in the Proteome of A. thaliana and S. cerevisiae

It was predicted the AD activity of all proteins of the reference proteome of A. thaliana (Colombia ecotype) and S. Cerevisiae (strain S288C) which was obtained from TAIR (Araport11) and SGD (S288C Genome release 64-3-1), respectively. Both proteomes with associated predictions are available in Supplementary data file 1 and can be loaded using Load_predictions_SI_data1.ipynb. It was predicted the secondary structure of every full-length protein using S4PRED and their structural disorder with IUPRED3 (long and short mode) (56, 57). Then the protein sequences and structural predictions were tiled into consecutive 53 amino acid tiles and predicted their AD activity using the PADDLE API for Python as described (1). All predictions in Python v3.9.5 with associated APIs were run and the pipeline is available in the Supplementary Data package. As one wanted to focus on tiles from non-TF genes, the TF databases PlantTFDB v5.0 and Yeastract+ was utilized to filter out any tiles derived from TFs. Tiles from genes that achieved a PADDLE predicted activation >30 were selected, yielding 12,000 A. thaliana tiles and 6,000 S. cerevisiae tiles with a dynamic range of PADDLE predicted activation strength between 17 and 138.

### Plasmid Library Construction

The library of both Arabidopsis and Saccharomyces ADs were generated by mapping the tiles back to their native DNA sequence in the respective reference genomes, retrieved from TAIR and SGD (all sequences in SI Table 1). 18,000 unique DNA oligos coding for 53 amino acid long putative activators were synthesized in one oligo pool by Twist Bioscience. Each oligo contains a 24 bp upstream primer (GCGGGCTCTACTTCATCGGCTAGC) (SEQ ID NO:100), 159 bp encoding the activator candidate, a 21 bp primer (TGATAACTAGCTGAGGGCCCG) (SEQ ID NO:101) with four stop codons in 3 frames and the Apal site. Specifically, 75 ng of template and 12 rounds of PCR in 16 parallel 50 μL reactions using primers LC3.P1_Lib_Hom_up_5′ (GCGGGCTCTACTTCATCGGCTAGC) (SEQ ID NO:102), which adds homology arms and YL_randBCs_R1_3′ which adds random 11 nt barcodes and downstream homology arms (NEB Q5 polymerase Tm=70C) were used. The PCR product was pooled and cleaned using the Monarch PCR and DNA kit, followed by product visualization on a 1% Agarose gel. Vector pMVS219 was linearized using NheI, AscI and PacI and used for library assembly. The assembly was performed using 100 ng of linearized backbone and 7.5 ng of PCR product using NEB Hifi DNA Assembly Master Mix in 8 10 μL reactions. Assemblies were electroporated into DH5β cells (NEB C3020K), and >1,000,000 colonies were recovered.

The plasmid sequence of the library assembly vector pMVS219 is available in the Supplemental Information (which is available at the website for: cell.com/cell-systems/fulltext/S2405-4712(24)00151-0), which is incorporated by reference.

### Primers:

YL_randBCs_R1_3′
(SEQ ID NO: 103)
AATTCGCTTATTTAGAAGTGGCGCGCCNNNNNNNNNNNCGGGCCCTCA
GCTAGTTATCA
LC3.P1_Lib_Hom_up_5′
(SEQ ID NO: 104)
GCGGGCTCTACTTCATCGGCTAGC

### Yeast Library Construction and Measurement

To ensure singular constructs per cell, the library was introduced into the URA3 locus of strain DHY211 (MATα, MKT1(30G) RME1(INS-308A) TAO3(1493Q), CAT5(91M) MIP1(661T), SAL1⁺ HAP1⁺). Employing the established yeast transformation method (58), the transformation was subjected to 30 minutes at 30° C. followed by 60 minutes at 42° C. To minimize potential PCR errors, SalI and EcoRI digestion was performed on the plasmid library, releasing the section encompassing the ACT1 promoter, the synthetic TF, and the KANMX marker. Simultaneously, PacI digestion was conducted to cleave plasmids devoid of an activation domain variant and barcode insert, thereby reducing the occurrence of transformants with inactive TFs. Directed integration into the URA3 locus was guided by 500 bp upstream homology spanning the URA3 and ACT1 promoters, along with a corresponding 500 bp downstream homology region spanning the TEF and URA3 terminators. Transformation utilized a molar ratio of 1:3 for linearized library to homology arms, with 28 μmol of linearized library per reaction. The transformed library was plated on YPD, followed by an overnight incubation at 30° C., and subsequent replica-plating onto freshly prepared SC G418 plates. Employing this process across 80 reactions yielded an estimated >1,000,000 individual colonies. Subsequently, the transformants were collectively mated with an FY5 strain containing the reporter integrated into the uncertain ORF, YBR032w. Diploids were selected on YPD with G418 (200 μg/ml) and NAT (100 μg/ml) (strain MY436 P3::GFP NAT @YBR032w S288C NAT-R), resulting in prototrophic diploids. These 110,000 yeast transformants were mated in batches, and prior to the final experiment, batches were pooled and multiple aliquots were frozen.

### Fluorescence Activated Cell Sorting and Library Preparation

Each sorting experiment was preceded by thawing a frozen glycerol stock, followed by overnight growth in SC+G418+NAT. Cultures were cultivated in synthetic complete (SC) dextrose media at 30° C. (59). Prior to fluorescence-activated cell sorting (FACS), overnight cultures were diluted (1:5) into SC+1 μM ß-estradiol and incubated for 3.5-4 hours at 30° C. The yeast library was sorted on a Aria-fusion cell sorter at the UC Berkeley Flow Cytometry core facility. The parent yeast strain was used with the reporter and a TF lacking an activation domain as a negative control to determine autofluorescence and baseline mCherry levels. 1 million cells of the synthetic TF library were sorted into 8 bins with each bin roughly covering 11% of the entire observable population in the GFP channel. To test reproducibility another 500,000 cells were sorted into the same bins.

Sorted cells were grown overnight in SC at 30° C. and gDNA was extracted with the Zymo YeaSTAR (#D2002) kit. Barcodes were amplified by PCR (CP21.P14, CP17.P12 NEB Q5 for 20 cycles, Tm 67° C.). Phasing nucleotides as well as overhangs for indexing primers using primer mixtures SL5.F[1-4] and SL5.R[1-4](NEB Q5 for 20 cycles, Tm 62° C.) were added. Finally dual indexing primers using the i5 and i7 system from Illumina (NEB Q5 for 20 cycles, Tm 65° C.) were added. Then a bead cleanup was performed. The library was sequenced on an Illumina Novaseq 6000 system with 2×150 bp paired end reads.

The library performance was assessed against known ADs from GCN4 and VP16 on a BD Accuri™ C6 flow cytometer (BD Biosciences). All strains were grown in SC+G418+NAT at 30 overnight and diluted (1:5) into SC+/−1 μM ß-estradiol and incubated for 3.5-4 hours at 30° C. Samples were washed with cold 1×PBS (137 mmol NaCl, 2.7 mM KCl, 1.8 mM KH₂PO₄, 10 mM Na₂HPO₄) once before measurement. Per sample 100,000 events were recorded and analyzed using the Python fcsparser package.

### Plant Experiments

Generated binary vectors were transformed into Agrobacterium tumefaciens strain GV3101. Selected transformants were inoculated in liquid media with appropriate selection the night before the experiment. A. tumefaciens strains were grown until OD₆₀₀ between 0.8 and 1.2 and were mixed equally (final OD₆₀₀=0.5 for each strain) with the strain harboring the assay reporter construct to a final OD₆₀₀=1.0. Cultures were centrifuged for 10 min at 4000 g and resuspended in infiltration buffer (10 mM MgCl₂, 10 mM MES, and 200 μM acetosyringone, pH 5.6). Cultures were induced for 2 h at room temperature on a rocking shaker. Leaves 6 and 7 of 4 week old N. benthamiana plants were syringe infiltrated with the A. tumefaciens suspensions. Post infiltration N. benthamiana plants were maintained in the same growth conditions as described above. Leaves were harvested three days post infiltration and 16 leaf disks from two leaves and 3 plants total per construct were collected. The leaf disks were floated on 200 μL of water in 96 well microtiter plates and GFP (Ex. λ=488 nm, Em. λ=520 nm) and RFP (Ex. λ=532 nm, Em. λ=580 nm) fluorescence measured using a Synergy 4 microplate reader (Bio-tek). The reporter construct for the screen was pms6370 containing GFP and dsRed expression cassettes. GFP expression was driven by a fusion of five previously characterized GALA binding sites with the core WUSCHEL promoter (60). GFP expression was normalized using dsRed driven by the constitutive MAS promoter on the same plasmid.

### Analysis of Barcodes and Inferring Activity

After demultiplexing samples, only the reads that contained a perfect match to a designed tile were kept. For each set of 8 sorted samples, two normalizations were performed. The reads by the total number of reads in each bin were first normalized. Then each designed tile was normalized across the 8 bins to calculate a relative abundance. Then relative abundances to an activity score for each tile were converted by taking the dot product of the relative abundance with the median fluorescence value of each bin. This computation is a weighted average. Tiles with less than 10 reads were not included in the final dataset. Later post hoc analysis suggested that tiles with at least 1000 reads were well measured.

During plasmid library construction random barcodes were added to the designed tiles. To build a map linking designed tiles to barcodes, all of the sequencing data from the 32 sorted samples [and a sequencing library created from the plasmid pool] were combined. This map is used to compare two modes of analysis. First, for the primary analysis used in the manuscript, only the tile sequences is used, effectively combining all the barcodes together and ignoring independent transformations. Second, the analysis is repeated for each AD+barcode combination, in effect measuring the activity of each independent transformant of each tile. The methods largely agreed very well.

### Quality Control of Activity Data

The data are of high quality. Compared to the original method, a novel barcoding strategy is used to randomly assign 100s of barcodes to each fragment and recovered an order of magnitude more unique integrations. The increased complexity of the library meant that soring 1e6 cells per bin introduced a significant bottleneck for reliably measuring each integration. However, the multiple independent integrations for each designed tile allowed one to measure variability in activity in a new way.

### Data Analysis

The data and underlying sequences of the tiles were analyzed and visualized using the following APIs in Python v3.9.5: pandas, seaborn, matplotlib, numpy and scipy. All associated Jupyter Notebooks for producing all Figures are in the Supplementary Data files (which is available at the website for: cell.com/cell-systems/fulltext/S2405-4712(24)00151-0), which is incorporated by reference. The library was sorted by activity and split it into four equal sized quartiles with 4388 tiles per quartile. To gauge the composition of each tile in each quartile, the amino acid frequencies of all amino acids in each tile was calculated. For the amino acid density analysis a sliding window size 5 was applied along every position of each tile, averaging the frequencies of amino acid occurrence of each aminoacid for each quartile. The amino acid window size to be 5 was chosen to not bias the analysis for short AD motifs like the 9aaTAD (61). Then the amino acid frequencies were grouped based on functional groups which was defined as follows: acidic (D, E) and hydrophobic (W, L, F, Y). To gauge the disorder of tiles the disorder predictor Metapredict which integrated the outcomes of multiple independent disorder predictors was utilized (62). Confidence intervals were calculated using the seaborn pointplot function.

The single amino acid resolution of the tiling experiments was utilized to gauge the effect on AD activity when one C-terminal amino acid is gained or one N-terminal amino acid is lost. A subset of tiles only including tiles that had at least one consecutive neighboring tile, meaning a pair of identical tiles with only one amino acid difference in the C- and N-terminus was generated. From this subset was calculated the change of AD activity between consecutive pairs of tiles and associated the lost and gained amino acid during the step. The analysis was performed for the entire library independent of whether a tile was defined as an AD or not.

### Mapping Putative Coactivators

To generate a map of putative coactivators in both plants and yeast one firstly subdivides the library into genes with and without tiles with AD activity. Hence, only parent genes of tiles in the 50% of the library that had higher activity were studied. Then parent genes were further subgrouped into nuclear-genes by utilizing SUBA5 for Arabidopsis and YeastGFP/YPL+. To gauge their function Gene Ontology was used. For yeast the SGD GO term slim mapper and for Arabidopsis the functional categorization of GO terms in the bulk data retrieval tool were used. Then the molecular functions linked to all nuclear genes in both species were manually studied to find known coactivators and chromatin regulators. Then all known coactivators excluded from the list was used to generate the final map of putative coactivators. For non-nuclear genes the same approach was used.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A synthetic transcription factor (TF) comprising (a) a DNA-binding domain of a transcription factor linked to (b) an activator domain (AD) comprising 50 to 55 amino acid residues, and having (i) one or more acidic amino acid residues in one or more positions within positions 33 to 46; (ii) one or more disorder promoting amino acid residues in one or more positions within positions 1 to 5; (iii) one or more hydrophobic amino acid residues in one or more positions within positions 27 to 40, or positions 30 to 36; and (iv) one or more order promoting amino acid residues in one or more positions within positions 15 to 43, positions 18 to 34, positions 21 to 33, or positions 25, 29, 30, 31, or 32.

2. The synthetic TF of claim 1, wherein each of the acidic amino acid residue is independently D or E.

3. The synthetic TF of claim 1, wherein each of the disorder promoting amino acid residue is independently A, E, G, K, Q, S, or P.

4. The synthetic TF of claim 1, wherein each of the hydrophobic amino acid residue is independently W, L, F, or Y.

5. The synthetic TF of claim 1, wherein each of the order promoting amino acid residue is independently C, F, H, I, L, M, N, V, W, or Y.

6. The synthetic TF of claim 1, wherein the AD comprises a sequence of any one of SEQ ID NO:1-55.

7. The synthetic TF of claim 1, wherein the synthetic TF further comprises (c) a nuclear localization sequence (NLS).

8. The synthetic TF of claim 1, wherein the DNA-binding domain is a deactivated RNA-guided nuclease variant of Cas9 (dCas9).

9. A nucleic acid encoding the synthetic TF of claim 1.

10. A nucleic acid encoding an activator domain comprising an amino acid sequence of any one of SEQ ID NO:1-55.

11. A vector comprising the nucleic acid of claim 10.

12. A host cell comprising the vector of claim 12, wherein the host cell is capable of expressing the synthetic TF or activator domain.

13. A system comprising a nucleic acid of claim 10 and a second nucleic acid, or the nucleic acid, encodes a gene of interest (GOI) operatively linked to a promoter and one or more activator binding domains, wherein the synthetic TF binds at least one of the one or more activator binding domain such that the synthetic TF modulates the expression of the GOI.

14. A genetically modified eukaryotic cell or organism comprising: (a) (i) one or more nucleic acids each encoding one or more transcription activators operatively linked to a promoter; and (b) one or more nucleic acids each encoding one or more independent genes of interest (GOI) each operatively linked to the promoter that is activated by the one or more transcription activators; wherein at least one transcription activator is a synthetic transcription factor (TF) of claim 1.
